(19) United States
(12) Patent Application Publication
(10) Pub. No.: US 2024/0309391 A1
(43) Pub. Date: Sep. 19, 2024

(54) COMPOSITIONS, METHODS, AND SYSTEMS FOR ENHANCED DNA TRANSFORMATION IN BACTERIA

(71) Applicant: North Carolina State University, Raleigh, NC (US)

(72) Inventors: Justin M. Vento, Raleigh, NC (US); Chase Lawrence Beisel, Raleigh, NC (US)

(21) Appl. No.: 18/578,558

(22) PCT Filed: Jul. 13, 2022
(86) PCT No.: PCT/US22/73690

§ 371 Date: Jan. 11, 2024

Related U.S. Application Data
(60) Provisional application No. 63/222,020, filed on Jul. 15, 2021.

(51) Int. Cl. C12N 15/74; C12N 9/10; C12N 15/10; C12N 15/70

(52) U.S. Cl. CPC C12N 15/74; C12N 9/1007; C12N 15/1065; C12N 15/70; C12N 15/746

(57) ABSTRACT

The present disclosure provides compositions, methods, and kits related to DNA transformation in bacteria. In particular, the present disclosure provides novel compositions and methods for enhancing DNA transformation by replicating a DNA methylation pattern used in a bacterial host strain. The compositions, methods, and systems described herein utilize cell-free transcription-translation mixtures and DNA methylation complexes to increase transformation efficiency and efficacy for any bacterial host.

### CROSS REFERENCE TO RELATED APPLICATIONS

This application claims priority to and the benefit of U.S. Provisional Patent Application No. 63/222,020 filed Jul. 15, 2021, which is incorporated herein by reference in its entirety for all purposes.

### GOVERNMENT FUNDING

This invention was made with government support under grant number MCB1452902 awarded by the National Science Foundation. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: 21,011 Byte (XML) file named “NCSU-39621-601” created on Jul. 12, 2022.

### FIELD

The present disclosure provides compositions, methods, and kits related to DNA transformation in bacteria. In particular, the present disclosure provides novel compositions and methods for enhancing DNA transformation by replicating a DNA methylation pattern used in a bacterial host strain. The compositions, methods, and systems described herein utilize cell-free transcription-translation mixtures and DNA methylation complexes to increase transformation efficiency and efficacy for any bacterial host.

### BACKGROUND

The study and manipulation of bacteria is a central part of basic research, biotechnology, agriculture, and human health. In basic research, bacteria are found in virtually every habitable environment and exhibit wide ranging genetic architectures, metabolisms, and physiologics that are being widely studied. Many of these metabolic and physiological capabilities are also being exploited within industrial biotechnology, such as for chemical overproduction. Many naturally occurring bacteria associate with plants, creating opportunities to enhance agriculture through these bacteria. Finally, bacteria are closely associated with human health, whether as pathogens that cause disease or as members of the human microbiota that have been linked to health benefits as well as various metabolic, autoimmune, and neurological health conditions. Probiotic and commensal bacteria can also be engineered as non-invasive recorders of health status, as vaccines, or as cell-based therapies. Being able to genetically manipulate these bacteria is essential in all of these areas.

One of the key steps to genetic manipulation is transforming a bacterium with DNA. The transformed DNA can express genes-of-interest or be used to modulate gene expression or alter the genome of the bacterium. DNA can also be introduced into a bacterial cell through different means, such as electroporation, chemical competence, natural competence, phage delivery, conjugation, and nanoparticle delivery. Once DNA is in the cell though, it must circumvent a battery of defense systems meant to eliminate foreign DNA associated with mobile genetic elements such as phages. One of the most prominent defense systems in bacteria are restriction-modification (R-M) systems. These systems assess the methylation state of all DNA in the cell and cut DNA with the incorrect pattern. In general, a DNA methyltransferase (MTase) methylates specific DNA sequences at a particular location, while a restriction endonuclease (REase) cleaves at sequences either with or without a specific methylation. The exact configuration of these systems varies across the different types, such as Type I systems that rely on a specificity protein to direct the separate MTase and restriction REase to a particular site, while Type II systems encode specificity within the MTase and REase. These systems can be so efficient that an introduced DNA with the wrong pattern is cleaved immediately in numerous locations, resulting in massive drops in the apparent transformation efficiency. Because these patterns vary between strains, isolating plasmid DNA with the correct pattern is extremely difficult to create, particularly for any randomly selected bacterium.

Different methods of bypassing R-M systems to boost DNA transformation efficiency have been developed in bacteria to address this challenge. The most direct technique is to delete R-M genes responsible for sequence recognition and restriction. However, this technique is limited to strains with an adequate level of prior DNA transformation to permit homologous recombination. If DNA motifs recognized by R-M systems are well characterized, they can be systematically removed from transformed plasmids to bypass restriction. This approach is effective but requires extensive characterization for each new strain, and the required mutations to the plasmid could be problematic (e.g., within a resistance gene or the origin-of-replication). The most common method of bypassing R-M systems is to alter the methylation pattern of the plasmid prior to transformation to match the host's methylome. For some strains, this can be done in vitro if commercial MTases are available that methylate similarly to the host MTases, by incubating DNA with purified host MTases, or a cell lysate prepared from cells containing active host MTases. However, the most common approach is to methylate a plasmid in vivo by passaging it through an E. coli strain expressing some of the host MTases. While this approach successfully boosted transformation in several species, it is time intensive to set up for each new strain and often fails due to inefficient methylation or toxicity in s coil, especially when multiple MTases are required. For instance, many strains can contain upwards of 10 MTases, yet only two can be reasonably expressed in a given bacterium. Thus, there is a need for a widespread approach to rapidly boost DNA transformation across diverse bacteria.

One unexplored route involves cell-free transcription-translation systems (TXTL). These specially prepared lysates or reconstituted systems recapitulate transcription, translation, and some aspects of metabolism. As a result, adding DNA encoding genes-of-interest results in the production of active RNAs and proteins. Traditionally. TXTL was used for producing individual proteins considered cytotoxic, such as inhibitors of DNA replication or cell wall synthesis. More recently TXTL has been used for characterizing gene circuits, performing simple metabolic reactions, and for producing on-demand vaccines. However, they have not been harnessed as a means for actively modifying added components. Expressing MTases that create DNA modifications known to alter gene expression, in some cases proving cytotoxic, could also pose a major challenge for TXTL. Nonetheless, TXTL offers a unique opportunity to express one or even multiple MTases at a time without the need for protein purification or expression in E. coli. As disclosed further herein, it was surprisingly found that TXTL systems offered a robust platform for methylating added DNA, whether with one or multiple MTases, and the resulting methylated DNA significantly enhances transformation efficiency. In accordance with these findings, a high-throughput strategy for identifying MTases for a given host strain was developed. This approach represents a broadly applicable platform for enhancing transformation that overcomes challenges associated with current approaches.

### SUMMARY

Embodiments of the present disclosure include a composition for methylating target DNA for transformation into a host cell. In accordance with these embodiments, the composition includes a cell-free transcription-translation mixture and at least one expression construct encoding one or more components of a methylation complex. In some embodiments, the methylation complex replicates at least a portion of a methylation pattern used by the host cell on the target DNA.

In some embodiments, replicating the methylation pattern used by the host cell results in enhanced transformation efficiency. In some embodiments, replicating the methylation pattern used by the host cell bypasses the host cell's restriction modification (RM) system.

In some embodiments, the host cell is a bacterial cell. In some embodiments, the bacterial cell is selected from the group consisting of an E. coli cell, a Bifidobacterium cell, a Bacillus cell, a Campylobacter cell, a Clostridium cell, a Corynebacterium cell, a Cyanobacterium cell, a Fusobacterium cell, a Geobacillus cell, a Helicobacter cell, a Klebsiella cell, a Lactobacillus cell, a Mycobacterium cell, a Neisseria cell, a Paenibacillus cell, a Prevotella cell, a Pseudomonas cell, a Ralstonia cell, a Salmonella cell, a Serratia cell, a Shewanella cell, a Staphylococcus cell, a Streptococcus cell, a Vibrio cell, and a Yersinia cell, or any variants thereof.

In some embodiments, the cell free transcription-translation mixture is derived from bacterial cell lysate. In some embodiments, at least one component of the cell free transcription-translation mixture is purified.

In some embodiments, one or more components of the methylation complex comprises at least one of: (i) a methyltransferase, and a specificity protein associated with a Type I RM system in the host cell; (ii) a methyltransferase associated with a Type II RM system in the host cell; (iii) a methyltransferase associated with a Type III RM system in the host cell; and/or (iv) an orphan methyltransferase.

In some embodiments, one or more components of the methylation complex comprises at least one methyltransferase. In some embodiments, at least one methyltransferase is derived from the host cell.

In some embodiments, at least one methyltransferase is mutated. In some embodiments, the mutated methyltransferase comprises a mutation that enhances methylation of the target DNA. In some embodiments, the mutation occurs in the N-terminal domain of a Type IA methyltransferase. In some embodiments, the mutated methyltransferase comprises a mutation that enhances recognition ofunmethylated DNA.

In some embodiments, the composition further comprises S-Adenosyl methionine (SAM) as a methyl donor. In some embodiments, the composition further comprises a suitable methylation buffer. In some embodiments, the composition further comprises RNase A and/or Proteinase K.

Embodiments of the present disclosure also include a kit for methylating target DNA for transformation into a host cell. In accordance with these embodiments, the kit includes a cell-free transcription-translation mixture and at least one expression construct encoding one or more components of a methylation complex. In some embodiments, the methylation complex replicates at least a portion of a methylation pattern used by the host cell on the target DNA.

In some embodiments of the kit, one or more components of the methylation complex comprises at least one of: (i) a methyltransferase, and a specificity protein associated with a Type I RM system in the host cell; (ii) a methyltransferase associated with a Type II RM system in the host cell; (iii) a methyltransferase associated with a Type III RM system in the host cell; and/or (iv) an orphan methyltransferase.

In some embodiments of the kit, the one or more components of the methylation complex comprises a mutated methyltransferase, wherein the mutation enhances recognition ofunmethylated DNA.

In some embodiments, the kit further comprises one or more of: (i)S-Adenosyl methionine (SAM) as a methyl donor; (ii) a suitable methylation buffer; (iii) RNase A; and/or (iv) Proteinase K.

In some embodiments, the kit further comprises a set of barcoded plasmid DNA constructs for determining a methylation pattern in a host cell.

In some embodiments, the kit further comprises a lookup table comprising a list of methyltransferases associated with a host cell.

In some embodiments of the kit, the host cell is selected from the group consisting of an E. coli cell, a Bifidobacteria cell, a Bacillus cell, a Campylobacter cell, a Clostridium cell, a Helicobacter cell, a Mycobacterium cell, a Neisseria cell, a Pseudomonas cell, a Salmonella cell, a Staphylococcus cell, a Streptococcus cell, a Vibrio cell, and a Yersinia cell, or any variants thereof.

Embodiments of the present disclosure also include a method of methylating a target DNA for transforming into a host cell. In accordance with these embodiments, the method includes expressing one or more components of a methylation complex in a cell-free transcription-translation mixture comprising the target DNA. In some embodiments, the methylation complex replicates at least a portion of a methylation pattern used by the host cell on the target DNA.

In some embodiments of the method, replicating the methylation pattern used by the host cell bypasses the host cell's restriction modification (RM) system and enhances transformation efficiency.

In some embodiments of the method, the host cell is selected from the group consisting of an E. coli cell, a Bifidobacterium cell, a Bacillus cell, a Campylobacter cell, a Clostridium cell, a Corynebacterium cell, a Cyanobacterium cell, a Geobacillus cell, a Helicobacter cell, a Klebsiella cell, a Lactobacillus cell, a Mycobacterium cell, a Neisseria cell, a Paenibacillus cell, a Prevotella cell, a Pseudomonas cell, a Ralstonia cell, a Salmonella cell, a Serratia cell, a Shewanella cell, a Staphylococcus cell, a Streptococcus cell, a Vibrio cell, and a Yersinia cell, or any variants thereof.

In some embodiments of the method, one or more components of the methylation complex comprises at least one of: (i) a methyltransferase, and a specificity protein associated with a Type I RM system in the host cell; (ii) a methyltransferase associated with a Type II RM system in the host cell; (iii) a methyltransferase associated with a Type III RM system in the host cell; and/or (iv) an orphan methyltransferase.

In some embodiments of the method, one or more components of the methylation complex comprises a mutated methyltransferase, wherein the mutation enhances recognition of unmethylated DNA.

In some embodiments, the method further comprises isolating and/or purifying the methylated target DNA prior to transforming into the host cell.

In some embodiments, the method further comprises determining the one or more components of a methylation complex that are compatible with the host strain.

In some embodiments of the method, determining the one or more components of a methylation complex that are compatible with the host strain comprises at least one of: (i) consulting a lookup table comprising a list of methyltransferases associated with the host cell; (ii) use of a set of barcoded plasmid DNA constructs for determining the methylation pattern used in the host cell, and/or (iii) conducting bioinformatics analysis.

Embodiments of the present disclosure also include a method of identifying a methyltransferase compatible with a host cell. In accordance with these embodiments, the method includes: (i) generating a library of plasmid DNA constructs, wherein at least one plasmid in the library comprises a barcode associated with a candidate methyltransferase or set of methyltransferases; (ii) expressing each of the candidate methyltransferase or set of methyltransferases in a cell-free transcription-translation mixture comprising the associated barcoded plasmid construct from the library of DNA plasmid constructs, thereby methylating the associated barcoded plasmid construct; (iii) transforming the library of barcoded and methylated plasmid DNA constructs into a host cell; and (iv) determining the frequency of each of barcode and methylated plasmid DNA construct isolated from the host cell, thereby identifying the candidate methyltransferase or set of methyltransferases compatible with the host cell.

In some embodiments, the host cell is selected from the group consisting of an E. coli cell, a Bifidobacterium cell, a Bacillus cell, a Campylobacter cell, a Clostridium cell, a Corynebacterium cell, a Cyanobacterium cell, a Geobacillus cell, a Helicobacter cell, a Klebsiella cell, a Lactobacillus cell, a Mycobacterium cell, a Neisseria cell, a Paenibacillus cell, a Prevotella cell, a Pseudomonas cell, a Ralstonia cell, a Salmonella cell, a Serratia cell, a Shewanella cell, a Staphylococcus cell, a Streptococcus cell, a Vibrio cell, and a Yersinia cell, or any variants thereof.

In some embodiments, the methyltransferase comprises at least one of: (i) a methyltransferase, and a specificity protein associated with a Type I RM system in the host cell; (ii) a methyltransferase associated with a Type II RM system in the host cell; (iii) a methyltransferase associated with a Type III RM system in the host cell; and/or (iv) an orphan methyltransferase.

Other aspects and embodiments of the disclosure will be apparent in light of the following detailed description.

### BRIEF DESCRIPTION OF THE DRAWINGS

FIG. 1: Representative schematic diagram illustrating the mechanisms by which Restriction-Modification (R-M) systems in bacteria distinguish self from non-self DNA by probing DNA methylation patters.

FIG. 2: Representative illustration of current approaches for overcoming R-M systems in bacteria and their corresponding limitations.

FIGS. 3A-3B: Representative schematic diagrams illustrating transcription-translation (TXTL)-based pipeline to imbue shuttle vectors with a host's methylation pattern to overcome restriction barriers in bacteria. Overview of the different types of R-M systems that cleave unmethylated DNA (FIG. 3A). MTases (darker shaded arrows) methylate DNA, while REases cleave unmethylated DNA (white arrows). Type I systems rely on a specificity protein (lighter shaded arrow) to bind the DNA recognition site. The cell-free pipeline to recreate host methylation patterns (FIG. 3B). MTases from the host strain are cloned into expression plasmids and individually expressed in TXTL. The expressed MTases are then combined to methylate initially unmethylated DNA that can then be purified and transformed into the host strain.

FIG. 4: Representative results demonstrating the use of the IMPRINT (Imitating Methylation Patterns Rapidly IN TXTL) method using E coil methyltransferases Dam and Dcm, according to one embodiment of the present disclosure. Assessing TXTL-based expression of Dam (encoded by M. EcoKdam) and Dcm (encoded by M.EcoKdcm) from F col MG1655. The REase DpnI cleaves sites methylated by Dcm, while the REase PspGI cleaves sites not methylated by Dcm. Restriction digestion of plasmid pJV24 methylated by Dam and/or Dcm using IMPRINT. HM: host-methylated plasmid DNA extracted from E col MG1655 and subjected to IMPRINT without any added MTases.

FIGS. 5A-5D: IMPRINT enhances DNA transformation of different plasmids in S. enterica LT2. R-M systems associated with S. enterica LT2. MTases in gray are considered orphan MTases that are not associated with REases (FIG. 5A). Number of MTase sites present in the two tested plasmids (FIG. 5B). Electroporation efficiency of plasmids pJV400 (top) and pJV414 (bottom) subjected to IMPRINT with different MTase combinations into S. enterica LT2 (FIG. 5C). All transformation efficiencies are normalized to that of unmethylated DNA. Error bars represent the geometric mean and standard deviation of triplicate independent experiments starting from separate TXTL reactions. The bottom of the bar marks the reference for statistical analysis. **: p<0.01. *: p<0.05. ns: p>0.05. HM: host-methylated plasmid DNA extracted from K coil MG1655 and subjected to IMPRINT without any added MTases.

FIGS. 6A-6C: Conserved Type IA R-M methyltransferase mutations boost methylation to achieve near-complete protection in S. enterica LT2. Aligned type IA MTases between E. coli and S. enterica (FIG. 6A). Mutations previously shown to accept unmethylated DNA substrates are shown in red. IA MTases normally prefer hemimethylated DNA as substrates. Restriction digestion of plasmid pJV412 methylated using IMPRINT (FIG. 6B). A HinFI restriction site was inserted to be methylated through one of the MS.SenLT2II recognition sites. HinFI digests the plasmid in six other locations, explaining the consistent lower bands on the gel. Electroporation efficiency of plasmid pJV400 subjected to IMPRINT with different MTase combinations into S entenca LT2 (FIG. 6C). All transformation efficiencies are normalized to that of unmethylated DNA. Error bars represent the geometric mean and standard deviation of triplicate independent experiments starting from separate TXTL reactions. The bottom of the bar marks the reference for statistical analysis. **: p<0.01. *: p<0.05. ns: p>0.05. HM: host-methylated plasmid DNA extracted from E. coli MG1655 and subjected to IMPRINT without any added MTases.

FIG. 7: Representative illustration of high-throughput IMPRINT (HT-IMPRINT) for determining optimal methylation patterns in recalcitrant Bifidobacteria. Barcodes are associated with each MTase combination to track how that combination impacts the transformation efficiency.

FIGS. 8A-8H: High-throughput IMPRINT determines optimal methylation patterns in recalcitrant Bifidobacteria. R-M systems associated with B. breve UCC2003 (FIG. 8A). Heat map of the relative impact of different MTase combinations using HT-IMPRINT with pJV420 in B. breve UCC2003 (FIG. 8B). Three barcodes were associated with each MTase combination, where the reported values are the median relative abundance normalized to the untransformed library. Electroporation efficiency of plasmid pJV420 subjected to IMPRINT with different MTase combinations into B. breve UCC2003 (FIG. 8C). R-M systems associated with B. longum ATCC 15707 (FIG. 8D). Heat map of the relative impact of different MTase combinations using HT-IMPRINT with pJV420 in B. longum ATCC 15707 (FIG. 8E). Two barcodes were associated with each MTase combination, where the reported values are the number of colonies possessing a barcode associated with the indicated MTase combination. Electroporation efficiency of plasmid pJV420 subjected to IMPRINT with different MTase combinations into B. longum ATCC 15707 (FIG. 8F). Compatibility between optimal MTase combinations and transformation efficiency across bacterial strains (FIG. 8G). The plasmid pJV420 was methylated with optimal patterns for each strain using IMPRINT and transformed into the different strains. E. coli EC135 lacks any R-M systems and thus serves as a transformation control. Electroporation efficiency of pJV420 with the different methylation patterns into the different bacterial strains (FIG. 8H). All transformation efficiencies for a given strain are normalized to that of unmethylated DNA. Heat maps represent the geometric mean of triplicate transformations. See FIG. 15 for individual measurements. Error bars in FIGS. 8C and 8F represent the geometric mean and standard deviation of triplicate independent experiments starting from separate TXTL reactions. The bottom of the bar marks the reference for statistical analysis. **: p<0.01. *: p<0.05. ns: p>0.05.

FIG. 9: Comparing methylation by M.EcoKdam expressed from a plasmid or from a linear DNA template. M.EcoKdam was either cloned into a methyltransferase expression plasmid or was amplified from E. coli MG1655 genomic DNA with primers including a 17 promoter and T500 terminator sequence. Separate TXTL reactions were performed with the two DNA templates. A T7 RNA polymerase expression plasmid pJV441 was added to the linear expression reaction along with the GamS protein to protect the DNA from degradation by RccBCD present in the TXTL lysate. Methylation reactions were then performed from the overnight TXTL reactions by adding plasmid pJV24, a commercial Dam methylation buffer, and methyl donor SAM. The plasmid was then purified from the reaction, and DpnI restriction digestion was performed to assess whether the plasmid was Dam-methylated. As a positive control, commercial Dam methyltransferase was used to methylate plasmid pJV24. The linear template leads to modest digestion by DpnI, indicating functional expression of the Dam from this template.

FIG. 10: Validation of methylation by S. enterica LT2 methyltransferases. a-b, M.SenLT2dam and M.SenLT2dcm methylation. To validate that IMPRINT could be used to methylate plasmids with methyltransferases from S. enterica LT2, M.SenLT2dam and M.SenLT2dcm were both expressed on plasmids, either in the same TXTL reaction (Co.) or in separate TXTL reactions (Se.). Methylation reactions were then set up with either one or both methyltransferases using plasmid pJV400. After plasmid cleanup, DpnI (FIG. 10A) or PspGI (FIG. 10B) restriction digestions were performed to test if the plasmid was either Dam- or Dcm-methylated, respectively. Methylation by orphan methyltransferase M.SenLT2IV (FIG. 10C). An IMPRINT expression construct harboring M.SenLT2I V was added to a TXTL reaction. Then, a methylation reaction was performed with plasmid pJV447 that harbored two NsiI/M.SenLT2IV motifs. Methylation was assessed by restriction digestion with NsiI that would not cleave DNA methylated by M.SenLT2IV. Methylation by Type III methyltransferase M.SenLT2I (FIG. 10D). M.SenLT2I was expressed in TXTL either by cloning the methyltransferase into an IMPRINT expression construct or by amplifying it from S. enterica LT2 genomic DNA using primers that included T7 promoter and T500 terminator sequences. A T7 RNA polymerase expression plasmid pJV441 was added to the linear expression reaction along with the GamS protein to protect the DNA from degradation by RecBCD present in the TXTL lysate. The methyltransferase reactions were used to methylate the plasmid pJV184 harboring an overlapping HinFI/M.SenLT2I motif. After plasmid purification, restriction digestion with HinFI was performed, which would cut plasmid pJV184 seven times if not methylated by M.SenLT2I but only six times if methylated by M.SenLT2I. HM: S. enterica LT2 host-methylated plasmid.

FIGS. 11A-11C: Incomplete methylation by M.SenLT2II. Series methylation of methyltransferases in S. enterica LT2 (FIG. 11A). To overcome poor multiplexed methylation, a series methylation reaction was set up where shuttle plasmid pJV400 was first methylated with MS.SenLT2II, then the plasmid was purified and a second IMPRINT reaction was performed to methylate pJV400 with remaining methyltransferases M.SenLT2I, M.SenLT2dam, and/or M.SenLT2dcm. The methylated plasmids were then transformed into S. enterica LT2, where fold change in CFU relative to unmethylated pJV400 was determined as well as a protection score relative to unmethylated and host-methylated (HM) pJV400 controls. Dots represent transformations from three separate IMPRINT reactions, and the bar represents the average fold change in CFU as well as the protection score. Time course experiment with M.SenLT2I and MS.SenLT2II (FIG. 11B). To assess how quickly complete methylation was achieved by R-M methyltransferases in S. enterica LT2, methylation reactions of 1-, 2-, or 4-hours were performed using either M.SenLT2I or MS.SenLT2II and shuttle plasmid pJV400. Methylated plasmids were then transformed into S. enterica LT2 along with unmethylated pJV400 and host-methylated pJV400 (HM). A fold change relative to unmethylated pJV400 was calculated, and a protection score was determined for each methylated plasmid relative to unmethylated and host-methylated controls. Dots represent transformations from three separate IMPRINT reactions, and the bar represents the average fold change in CFU as well as the protection score. Methylation by MS.SenLT2II is incomplete (FIG. 11C). In order to determine if MS.SenLT2II was completely methylating plasmid DNA, plasmid pJV412 harboring an overlapping HinFI/MS.SenLT2II motif was methylated with MS.SenLT2II alone or in combination with other S. enterica LT2 methyltransferases M.SenLT2I, M.SenLT2dam, and M.SenLT2dcm. After the plasmid was purified from the IMPRINT reactions, a HinFI restriction digestion was performed, which would cleave pJV412 seven times if the plasmid was not methylated by MS.SenLT2II and would only cleave pJV412 six times if the plasmid was methylated by MS.SenLT2II.

FIG. 12: Time-course experiment comparing methylation by MS.SenLT2II and the mutants MS.SenLT2II_L85Q and MS.SenLT2II_L113R. To assess how quickly complete methylation was achieved by type I R-M methyltransferase MS.SenLT2II and MS.SenLT2II_L85Q and MS.SenLT2II_L113R, methylation reactions of 2 hours or 4 hours were performed by combining each methyltransferase with shuttle plasmid pJV400. Methylated plasmids were then transformed into S. enterica LT2 along with unmethylated pJV400 and host-methylated pJV400 (HM). A fold change relative to unmethylated pJV400 was calculated, and a protection score was determined for each methylated plasmid relative to unmethylated and host-methylated controls. Dots represent transformations from three separate IMPRINT reactions, and the bar represents the average fold change in CFU as well as the protection score. The bottom of the bar marks the reference for statistical analysis. **: p<0.01. *: p<0.05. ns: p>0.05.

FIG. 13: IMPRINT methylates a shuttle plasmid with all three R-M methyltransferases from B. breve UCC2003. To assess whether IMPRINT could be used to methylate plasmids with methyltransferases from Bifidobacteria, three type III R-M methyltransferases from B. breve UCC2003 (M.BbrUI, M.BbrUII, and M.BbrUIII) were expressed in separate TXTL reactions and added either separately or in combination to methylate shuttle plasmid pJV420. For one sample, half the amount of methyltransferases were added to the methylation reaction to determine if this would improve plasmid cleanup from TXTL lysate without affecting methylation (“½” sample). Methylation by M.BbrUI was validated by restriction digestion of plasmid pJV420 with Kasi which would only cleave DNA not methylated by M.BbrUI. Methylation by M.BbrUII was validated by restriction digestion of plasmid pJV420 with SalI which would only cleave DNA not methylated by M.BbrUII. Methylation by M.BbrUIII was validated by restriction digestion of plasmid pJV420 with PstI which would only cleave DNA not methylated by M.BbrUIII. HM=host-methylated (pJV420 propagated in B. breve UCC2003).

FIG. 14: Barcode abundances when performing HT-IMPRINT in B. breve UCC2003. The 8 possible methylation patterns from B. breve UCC2003 were assigned three barcodes each on pJV420_D (blue=barcode 1, yellow=barcode 2, red=barcode 3), and the pool of methylated plasmids were transformed together into B. breve UCC2003. Amplicons containing the barcoded portion of pJV420_D from the pooled library prior to transformation (L), the library from the plated colonies (P), and the library from the liquid outgrowth (O) were sent for high-throughput amplicon sequencing, where the number of reads from each barcode was determined.

FIG. 15: Cross-transformations of optimally methylated plasmids from different strains. To determine whether the optimal methylation pattern determined from each tested strain (S. enterica LT2, B. breve UCC2003, and B. longum ATCC15707) was specific to that strain only, a cross-transformation experiment was performed where shuttle plasmid pJV420 harboring each tested strain's optimal methylation pattern was used to transform the other strains. Plasmids that were unmethylated, methylated by MS.SenLT2II_L85Q and M.SenLT2I from S. enterica LT2, methylated by M.BbrUI and M.BbrUII from B. breve UCC2003, or methylated by M₂.Blo1217ORF1038 and MS₂.Blo1217ORF1481 from B. longum ATCC15707 were transformed into each strain, where E. coli EC135 was used as a control strain lacking R-M systems. The fold change in CFU was determined for each methylated sample relative to unmethylated pJV420. Dots represent the fold changes obtained from three individual transformations, and bars represent the average fold change.

FIG. 16: Diagram of a representative workflow that includes questions designed to determine whether and how IMPRINT can be performed, according to one embodiment of the present disclosure.

FIG. 17: Diagram of a representative workflow demonstrating the implementation of IMPRINT in conjunction with the workflow provided in FIG. 16, according to one embodiment of the present disclosure.

FIG. 18: Comparison of IMPRINT with other methods of bypassing R-M barriers to DNA transformation. Different methods of overcoming host R-M systems to boost transformation are compared, including plasmid artificial modification (PAM), methods involving in-vitro methylation by host cell lysates or purified methyltransferases, SyngenicDNA, and methods that involve deleted restriction endonucleases associated with R-M systems. Methods were compared based on how many R-M systems they could reasonably overcome, DNA compatibility, the average time the method takes to set up for a new strain, and any associated toxicity issues. Timing begins when DNA constructs for expressing MTases are available. No MTase constructs are needed for SyngenicDNA and KO R-M, so these start with the initial experimental procedures (methylome sequence and generating gene knockouts, respectively).

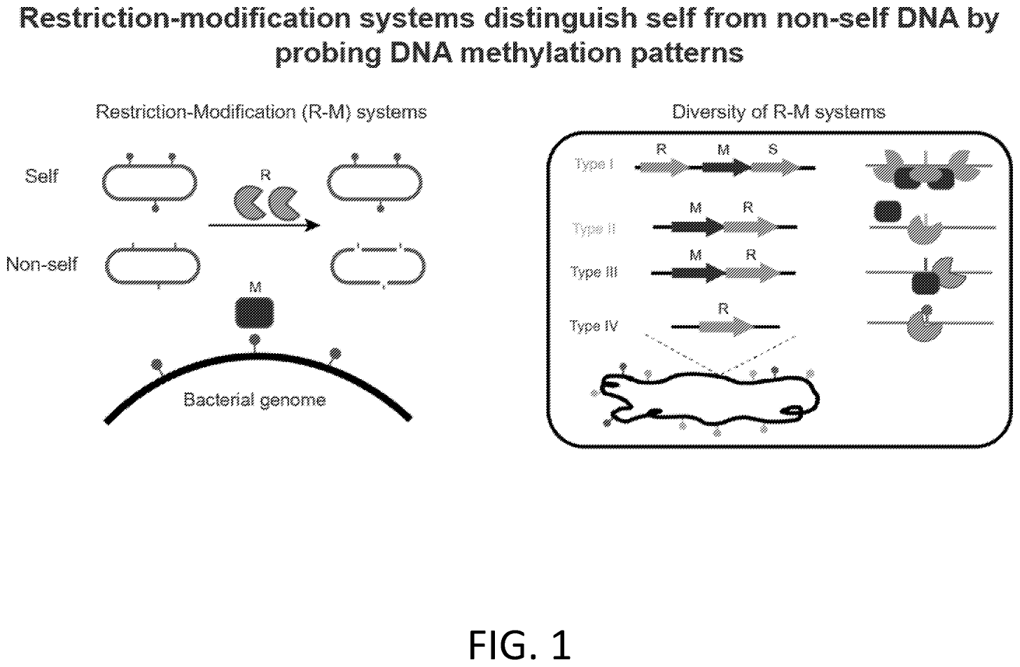

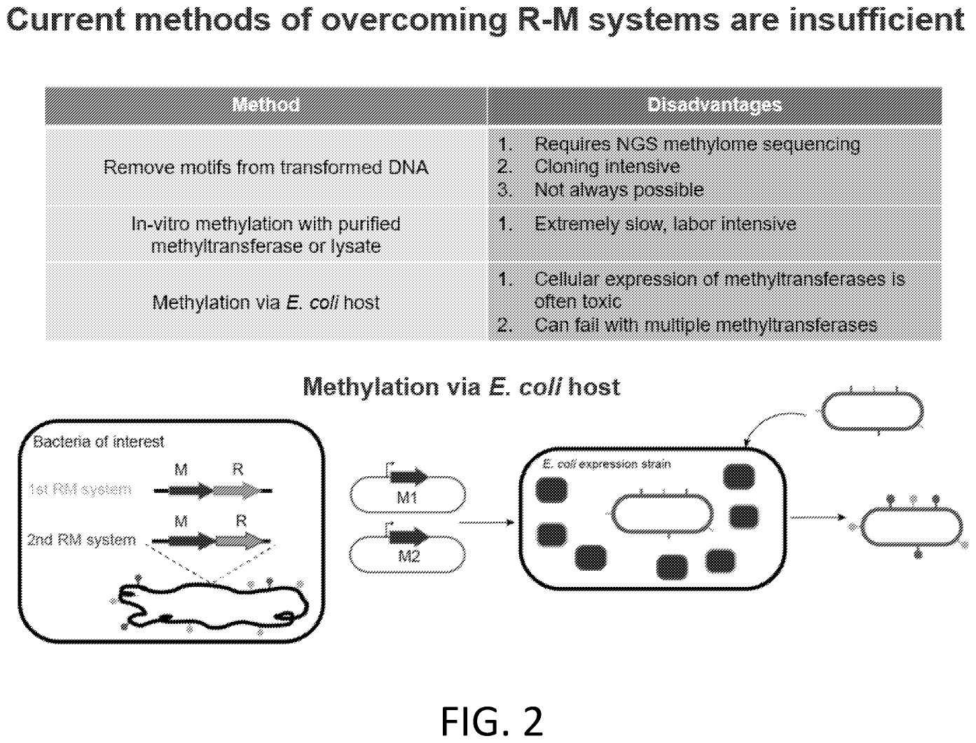

### DETAILED DESCRIPTION

The present disclosure provides compositions, methods, and kits related to DNA transformation in bacteria. In particular, the present disclosure provides novel compositions and methods for enhancing DNA transformation by replicating a DNA methylation pattern used in a bacterial host strain. The compositions, methods, and systems described herein utilize cell-free transcription-translation mixtures and DNA methylation complexes to increase transformation efficiency and efficacy for any bacterial host.

As described further herein, DNA transformation represents an ongoing and substantial challenge for those working with non-model bacteria. This step is critical for performing genetic manipulations, yet remains one of the most significant challenges for many bacteria. This is part due to the fact that the various ways to improve transformation developed for one bacterium do not necessarily transfer to even related strains. In many cases, the principal barrier is posed by restriction-modification systems that adorn host DNA with a specific methylation pattern and actively cleave DNA with a different pattern. Prior work has shown that mimicking a host's methylation pattern can radically boost DNA transformation. To date, creating this pattern has required expressing DNA methyltransferases (MTases) in E. coli or purifying the MTases to for in vitro reactions. However, only one or two MTases can be generally expressed in E. coli and many are cytotoxic, while MTase purification can be a laborious and time-consuming process. Additionally, these approaches have principally relied on Type II MTases encoded by a single protein, while other types require expressing multiple proteins. As a result, these approaches remain significant challenges and only partially replicate the methylation pattern.

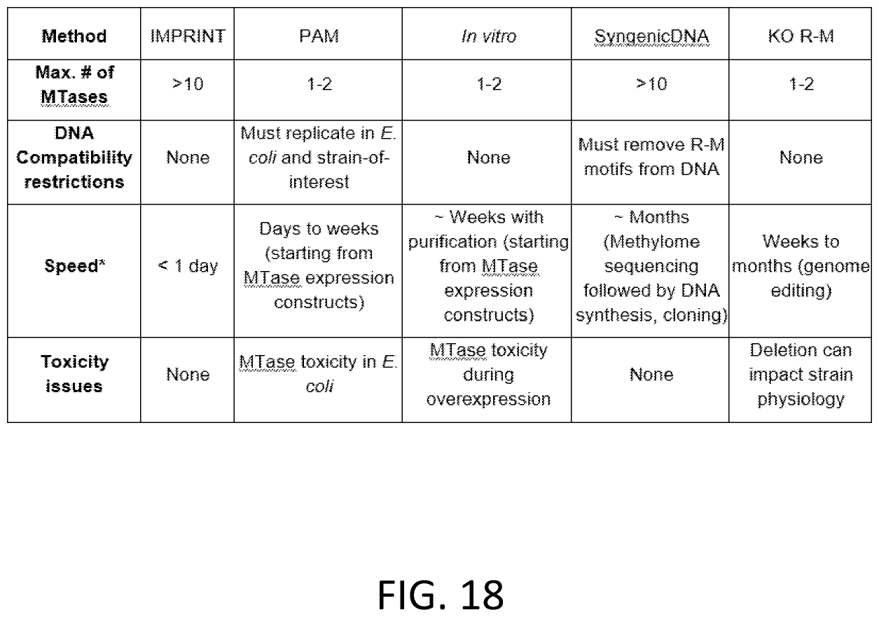

To address these issues, the IMPRINT (Imitating Methylation Patterns Rapidly IN TXTL) DNA transformation platform was developed. This method involves expressing MTases in a cell-free transcription-translation (TXTL) reaction mixture, and combining the expressed MTases in a methylation reaction with target plasmid DNA. The plasmid DNA can then be isolated and transformed into the target bacterium. The process that includes obtaining the MTase constructs and then the purified target plasmid DNA takes significantly less time than methods currently used, which can take days to weeks. In addition, TXTL reaction mixtures can be developed and used for all types of MTases and can be combined into a single methylation reaction. The time/costs savings and the ability to fully replicate a bacterium's methylation pattern represents a disruptive capability that would benefit bacterial researchers in biomedical, agricultural, and environmental sciences, as well as for strain engineering for cell-based therapies and industrial biotechnology (see, e.g., FIG. 18). Given that DNA methylation varies between even related strains, the compositions and methods of the present disclosure can be used to develop specific kits that are tailored to individual strains and corresponding services to identify and develop compatible methylation complexes for a given bacterium. In cases where transformation procedures that do not involve naked DNA are preferred (e.g., conjugation, phagemid delivery), IMPRINT can also be implemented to identify the most important MTases that should be expressed in the donor strain or packaging strain, respectively. These could represent the MTases that cause the greatest boost in transformation and/or can be expressed in a donor/packaging strain without severe cytotoxicity.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethvl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacctic acid, pseudouracil, queosine, 2-thiocytosine, and 2,6-diaminopurine.

The term “gene” refers to a nucleic acid (e.g., DNA) sequence that comprises coding sequences for the production of a polypeptide, precursor, or RNA (e.g., rRNA, tRNA, sRNA, microRNA, lincRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than about 300 residues long (e.g., between 15 and 100), however, as used herein, the term is also intended to encompass longer polynucleotide chains. Oligonucleotides are often referred to by their length. For example, a 24-residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

The term “homology” and “homologous” refers to a degree of identity. There may be partial homology or complete homology. A partially homologous sequence is one that is less than 100% identical to another sequence.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary,” “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs; cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure comprises a “double-stranded nucleic acid”. For example, triplex structures are considered to be “double-stranded.” In some embodiments, any base-paired nucleic acid is a “double-stranded nucleic acid.”

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Compositions and Methods of Use

As described further herein, embodiments of the present disclosure represent a novel way of increasing DNA transformation efficiency and efficacy in recalcitrant bacteria by quickly assessing and overcoming the DNA restriction barrier posed by host restriction-modification (R-M) systems. The compositions and methods provided in the present disclosure involve producing host methyltransferases using cell-free transcription-translation (TXTL) machinery from E. coli lysate and/or various purified TXTL components, and imbuing shuttle plasmids (e.g., target DNA) with the produced methyltransferases to rapidly methylate DNA before transforming it into the bacterial host. These compositions and method represent a stark contrast to the current state-of-the art methods for overcoming the R-M restriction barrier that require producing the desired methyltransferases inside an E. coli host, which is not only time intensive to set up, it also often results in a failed transformation due to induced cellular toxicity by the expressed heterologous methyltransferases.

The compositions and methods of the present disclosure are able to replicate rapidly the methylation patterns of a given bacterial host strain using a TXTL reaction mixture that involves three basic steps and takes approximately 18 hours to complete. In some embodiments, a first 12-14 hour TXTL reaction is set up to produce the desired methyltransferases (method of identifying a compatible methyltransferase are described further below). Then, a methylation reaction is conducted by combining the plasmid of interest with methylation buffer and a small amount of the TXTL mix containing the methyltransferases. A methyl donor (e.g., S-Adenosyl methionine or SAM) can be added to boost methylation, although it is not explicitly required because it is often times already present in the TXTL mixture. Finally, the target DNA plasmid is purified from the TXTL and methylation components using Proteinase K. RNase A, and column purification to yield clean, methylated DNA that is suitable for transformation into the bacterial host.

As described further herein, this method was demonstrated by expressing E. coli MG1655 Dam and Dcm methyltransferases, and validating complete methylation of a target plasmid with restriction digestion by methyl-sensitive restriction enzymes DpnI and PspGI. The method was then used to assess and quickly overcome the R-M barrier to DNA transformation in Salmonella enterica LT2, which involved expressing a Type III methyltransferase and Type I methyltransferase with the associated specificity protein from this strain. It was found that the Type I R-M methyltransferase does not efficiently utilize unmethylated DNA as a substrate, and improved methylation was achieved by mutating two amino acids in this methyltransferase that are conserved in the E. coli Type I R-M methyltransferase EcoK and have been found to promote methylation of unmethylated substrates. The method was then used to increase DNA transformation in the probiotic bacterium Bifidobacterium breve UCC2003 by expressing three well-characterized Type II R-M methyltransferases. As would be recognized by one of skill in the art based on the present disclosure, this method can be applied to any bacterial strains, including those of Bifidobacteria, that are currently difficult or virtually impossible to successfully transform with conventional methods/compositions.

Additionally, a high-throughput approach was developed to quickly determine the minimal methylation pattern for efficient DNA transformation in diverse Bifidobacteria strains by barcoding an E. coli-Bifidobacteria shuttle plasmid and sequencing a pool of plasmids from a single transformation containing every combination of methyltransferases. Overall, this approach provides a customized platform for rapidly improving and/or making feasible DNA transformation in any bacterium harnessing R-M defense systems.

In accordance with these embodiments, the present disclosure provides a composition for methylating target DNA for transformation into a host cell. In some embodiments, the composition includes a cell-free transcription-translation mixture and at least one expression construct encoding one or more components of a methylation complex. In some embodiments, the methylation complex replicates at least a portion of a methylation pattern used by the host cell on the target DNA. As demonstrated herein, replicating at least a portion of the methylation pattern used by a host cell on any given DNA plasmid results in enhanced transformation efficiency and efficacy as the methylated target DNA plasmid bypasses the host cell's restriction modification (RM) system.

As would be readily recognized by one of ordinary skill in the art based on the present disclosure, the IMPRINT methodology described herein can be applied to any bacterial host cell, and is particularly applicable for use in bacterial strains that are difficult to transform (e.g., the methylation patterns and/or methyltransferases in that host strain have not been characterized). For example, bacterial host strains that may be used with the IMPRINT methods of the present disclosure include, but are not limited to E. coli, Bifidobacterium, Bacillus, Campylobacter, Clostridium, Corynebacterium, Cyanobacterium, Geobacillus, Helicobacter, Klebsiella, Laclobacillus, Mycobacterium, Neisseria, Paenibacillus, Prevotella, Pseudomonas, Ralsionia, Salmonella, Serratia, Shewanella, Staphylococcus, a Streptococcus, Vibrio, and Yersinia, or any variants thereof.

In some embodiments, a composition for methylating target DNA for transformation into a host cell includes a cell-free transcription-translation (TXTL) reaction mixture that is derived from bacterial lysate, including any of the bacterial strains listed above. In some embodiments, at least one component of the cell free transcription-translation mixture is purified. That is, the TXTL mixtures of the present disclosure can include any combination of lysate and purified components, as long as the mixture (with or without a purified component) is able to facilitate the expression of a desired transcript into a functional protein. In other embodiments, such as those involving competent bacterial host strains, the cell free transcription-translation mixture is added directly to a host cell culture without purification.

As described further herein, the use of a transcription-translation mixture offers a unique method to methylate target DNA that is significantly easier, faster, and more scalable than existing approaches that rely on purified MTases or expressing MTases in F co/i. TXTL systems have been used to produce different types of proteins, although the proteins themselves represent the endpoint of the reaction, or these proteins or their products are purified for downstream applications. However, producing MTases using TXTL systems has not been explored for methylating DNA, and in particular, has not been explored for the purpose of enhancing DNA transformation. Therefore, prior to the results described in the present disclosure, the applicability of TXTL systems for DNA methylation applications was not known, including whether MTases could be sufficiently produced using TXTL systems to allow for methylation of target plasmid DNA. As described further herein, embodiments of the present disclosure demonstrate the utility of this approach.

In some embodiments, a composition for methylating target DNA for transformation into a host cell includes at least one expression construct encoding one or more components of a methylation complex. In some embodiments, the one or more components of the methylation complex includes at least one of a methyltransferase, and a specificity protein associated with a Type I RM system in the host cell. In some embodiments, the one or more components of the methylation complex includes a methyltransferase associated with a Type II RM system in the host cell. In some embodiments, the one or more components of the methylation complex includes a methyltransferase associated with a Type III RM system in the host cell. In some embodiments, the one or more components of the methylation complex includes an orphan methyltransferase. An orphan methyltransferase (e.g., Dam methylase) is a methyltransferase that is not part of a restriction-modification system but operates independently to regulate gene expression, mismatch repair, and bacterial replication amongst many other functions. In some embodiments, the one or more components of the methylation complex includes an orphan methyltransferase that is associated with a host cell or that is obtained from a different bacterium but produces the desired methylation pattern. In some embodiments, the one or more components of the methylation complex includes at least one methyltransferase. In some embodiments, the at least one methyltransferase is derived from the host cell.

As described further herein, existing approaches for overcoming R-M systems to facilitate DNA transformation in a bacterial host cell have generally been limited to Type II MTases, single proteins that methylate without other proteins, and usually only one or two of these MTases are used at a time. However, results of the present disclosure demonstrate the use of MTases from all types, and these be combined to methylate DNA with many MTases at one time (i.e., multiplexed). This facilitates the implementation of more complicated DNA methylation patterns that in turn greatly enhance transformation efficiency and efficacy compared to current approaches.

In some embodiments, methods and compositions of the present disclosure include using IMPRINT in a multiplexed platform (e.g., using multiple MTases). As described further herein, the identification of certain mutations in MTases can facilitate the use of multiple MTases in a given methylation reaction. In some embodiments, at least one methyltransferase is mutated. In some embodiments, the mutated methyltransferase comprises a mutation that enhances methylation of the target DNA. In some embodiments, the mutation occurs in the N-terminal domain of a Type IA methyltransferase. In some embodiments, the mutated methyltransferase comprises a mutation that enhances recognition ofunmethylated DNA.

In some embodiments, a composition for methylating target DNA for transformation into a host cell includes various other components. For example, in some embodiments, the composition comprises S-Adenosyl methionine (SAM) as a methyl donor. In some embodiments, the composition comprises a suitable methylation buffer. In some embodiments, the composition further RNase A and/or Proteinase K.

As would be recognized by one of ordinary skill in the art based on the present disclosure, IMPRINT can be used to enhance DNA transformation efficacy and/or efficiency in bacterial host strains that are cultured in isolation. However. IMPRINT compositions and methods can also be used with various other suitable delivery vehicles and/or devices to implement IMPRINT in other settings where genetic engineering can be advantageous, such as in situ microbiome editing, which includes but is not limited to, targeting bacteria in the environment, in the soil of agricultural fields, in human microbiomes, and the like (see, e.g., FIG. 18).

Additionally, IMPRINT can be used to enhance transformation with any DNA transformation methods, including conjugation (e.g., DNA transfer between bacteria through formation of a pilus) and phagemid delivery (e.g., delivery of a plasmid packaged by a bacteriophage particle). For example, as described further herein, bacteria can be engineered to express an MTase or a combination of MTases to facilitate the methylation of a plasmid DNA that can then be transferred via conjugation to another bacteria. IMPRINT is also compatible with any in vitro methylation reaction performed using an MTase or a combination of MTases. Furthermore, IMPRINT can be used with circular DNA as well as linear DNA. For example, circular and linear DNA can be used to express an MTase or a combination of MTases to perform IMPRINT, and circular and linear DNA can be methylated using IMPRINT to improve transformation efficiency.

In accordance with these embodiments, the present disclosure also includes a method of methylating a target DNA for transforming into a host cell. In some embodiments, the method includes expressing one or more components of a methylation complex in a cell-free transcription-translation mixture comprising the target DNA. In some embodiments, the methylation complex replicates at least a portion of a methylation pattern used by the host cell on the target DNA. In some embodiments of the method, replicating the methylation pattern used by the host cell bypasses the host cell's restriction modification (RM) system and enhances transformation efficiency.

In some embodiments of the method, the host cell includes, but is not limited to, E. coli, Bifidobacterium, Bacillus, Campvlobacter, Clostridium, Corynebacterium, Cyanobacterium, Geobacillus, Helicobacter, Klebsiella, Lactobacillus, Mycobacterium, Neisseria, Paenmbacillus, Prevotella, Pseudomonas, Ralstonia, Sallmonella, Serratia, Shewanella, Staphylococcus, a Streptococcus, Vibrio, and Yersinia, or any variants thereof.

In some embodiments of the method, one or more components of the methylation complex includes (i) a methyltransferase, and a specificity protein associated with a Type I RM system in the host cell; (ii) a methyltransferase associated with a Type II RM system in the host cell; (iii) a methyltransferase associated with a Type III RM system in the host cell; and/or (iv) an orphan methyltransferase. In some embodiments of the method, one or more components of the methylation complex comprises a mutated methyltransferase, wherein the mutation enhances recognition ofunmethylated DNA.

In some embodiments, the method further comprises isolating and/or purifying the methylated target DNA prior to transforming into the host cell. As would be recognized by one of ordinary skill in the art based on the present disclosure, methods for purifying a target DNA or plasmid prior to transformation into a bacterial host cell can include the use of a conventional DNA purification column, and/or treatment with RNase and/or Proteinase K.

### 3. Systems and Kits

Embodiments of the present disclosure also include a system or kit for methylating target DNA for transformation into a host cell. In accordance with these embodiments, the kit includes a cell-free transcription-translation (TXTL) mixture and at least one expression construct encoding one or more components of a methylation complex. In some embodiments, the methylation complex replicates at least a portion of a methylation pattern used by the host cell on the target DNA.

In some embodiments of the kit, one or more components of the methylation complex includes (i) a methyltransferase, and a specificity protein associated with a Type I RM system in the host cell; (ii) a methyltransferase associated with a Type II RM system in the host cell; (iii) a methyltransferase associated with a Type III RM system in the host cell; and/or (iv) an orphan methyltransferase.

In some embodiments of the kit, the one or more components of the methylation complex comprises a mutated methyltransferase, wherein the mutation enhances recognition ofunmethylated DNA. In some embodiments, the kit comprises one or more of: (i)S-Adenosyl methionine (SAM) as a methyl donor; (ii) a suitable methylation buffer; (iii) RNase A; and/or (iv) Proteinase K.

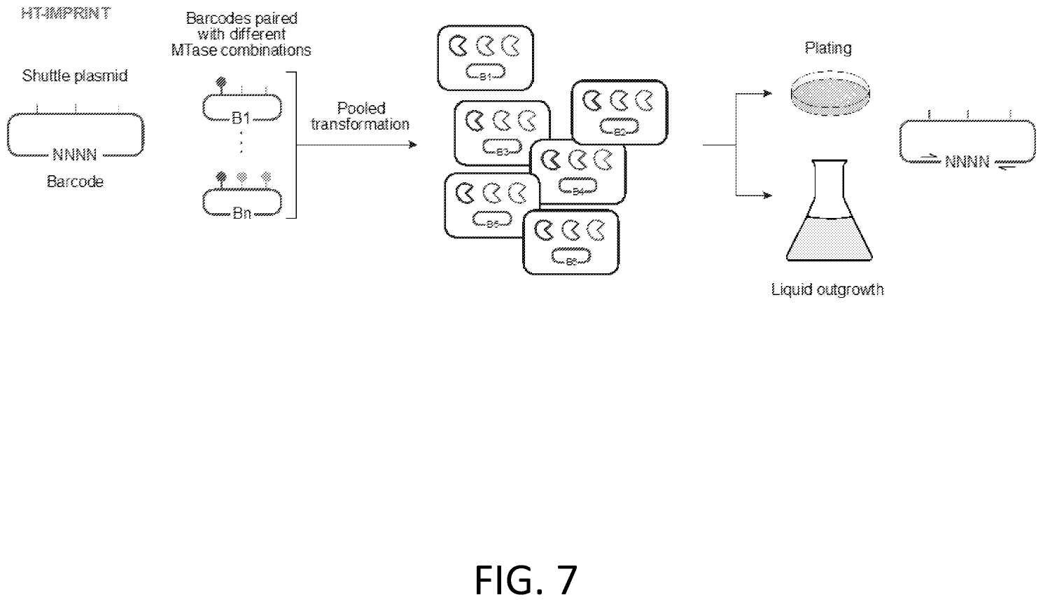

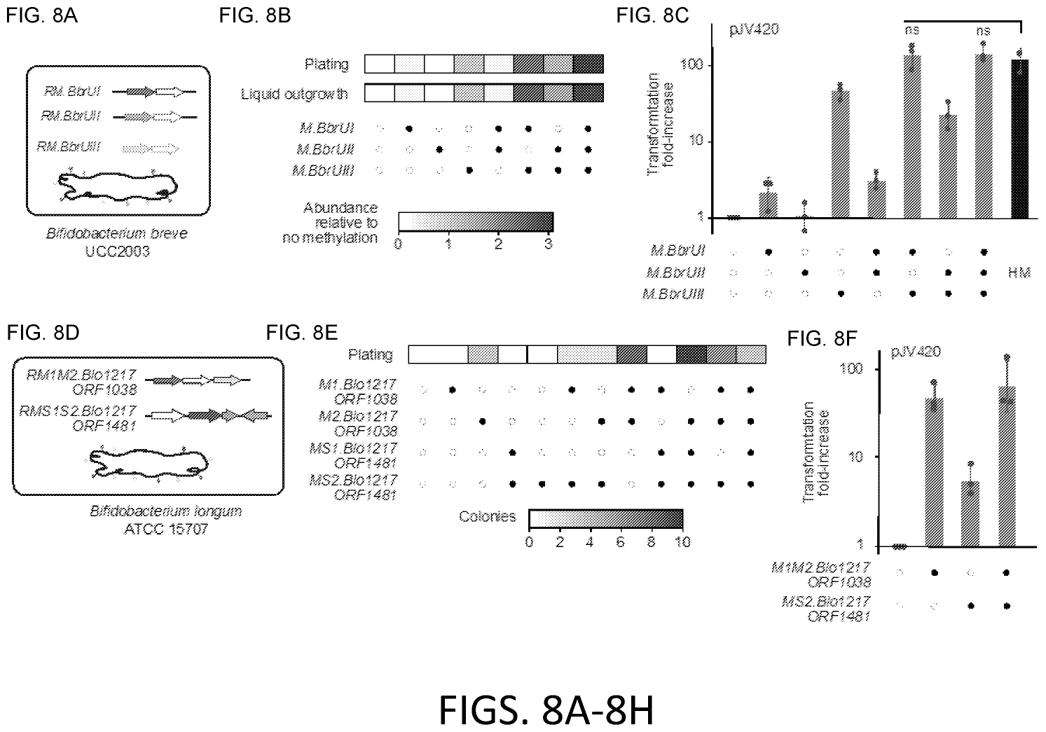

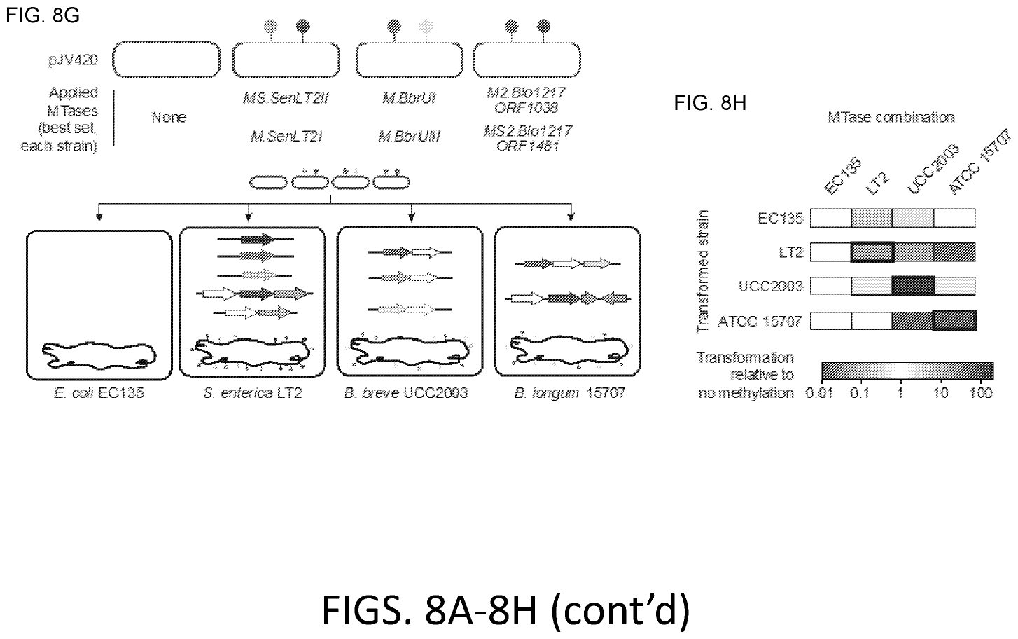

As described further herein, a kit for methylating target DNA for transformation into a host cell can include a set of barcoded plasmid DNA constructs for determining a methylation pattern in a host cell (see, e.g., FIGS. 7 and 8). In some embodiments, the kit further comprises a lookup table that includes a list of methyltransferases associated with a host cell, which allows a user to identify a particular methyltransferase to use with a given host strain. In some embodiments of the kit, the host cell includes, but is not limited to, E. coli, Bifidobacterium, Bacillus, Campylobacter, Clostridium, Corynebacterium, Cyanobacterium, Geobacillus, Helicobacter, Klebsiella, Lactobacillus, Mycobacterium, Neisseria, Paenibacillus, Prevotella, Pseudomonas, Ralstonia, Salmonella, Serratia, Shewanella, Staphylococcus, a Streptococcus, Vibrio, and Yersinia, or any variants thereof.

### 4. Methyltransferase Identification

As described further herein, embodiments of the present disclosure include methods of methylating a target DNA for transforming into a host cell, including expressing one or more components of a methylation complex in a cell-free transcription-translation mixture comprising the target DNA. In accordance with these embodiments, the present disclosure includes methods for determining whether one or more components of a methylation complex are compatible with a particular host strain. In some embodiments of the method, determining whether one or more components of a methylation complex are compatible with the host strain includes consulting a lookup table comprising a list of methyltransferases associated with the host cell. In some embodiments of the method, determining whether one or more components of a methylation complex are compatible with the host strain includes the use of a set of barcoded plasmid DNA constructs for determining the methylation pattern used in the host cell (see, e.g., FIGS. 7 and 8). In some embodiments, determining whether one or more components of a methylation complex are compatible with the host strain includes conducting bioinformatics analysis based on known features of MTases. Automated tools can also be used, such as that available through REBASE (see, e.g., rebase.neb.com/rebase/rebase).

In some embodiments, the method can be used to identify a methyltransferase that is compatible with a given host strain, such that it can be expressed as part of the IMPRINT methodology described herein, and used to enhance DNA transformation in that host cell. In some embodiments, one or more aspects of a method for identifying a suitable methyltransferase in a particular host strain can be performed by a user carrying out IMPRINT. In other embodiments, one or more aspects of a method for identifying a suitable methyltransferase in a particular host strain can be performed by an off-site scientific professional that supports a user that will carry out IMPRINT.

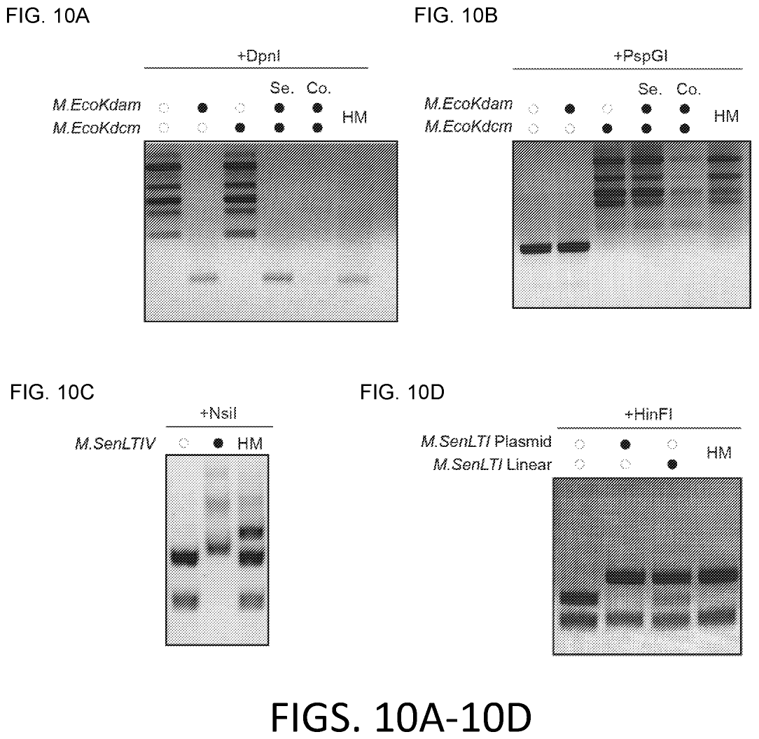

For example, a user that wishes to implement IMPRINT can provide answers to the questions in the workflow provided in FIG. 10. In some cases, a user can perform high throughput barcoding (see, e.g., FIG. 8 and Example 4) to determine the optimal methylation pattern for a plasmid they wish to transform into a particular host bacterial strain. In some cases, the user can then identify and clone the suitable methyltransferase that can replicate the methylation pattern to the degree required to enhance DNA transformation. In other cases, a user will consult a lookup table that comprises a list of suitable methyltransferases that will produce the desired methylation pattern. In still other cases, a user can conduct bioinformatics analysis on the genome of a particular host strain to identify a suitable methyltransferase.

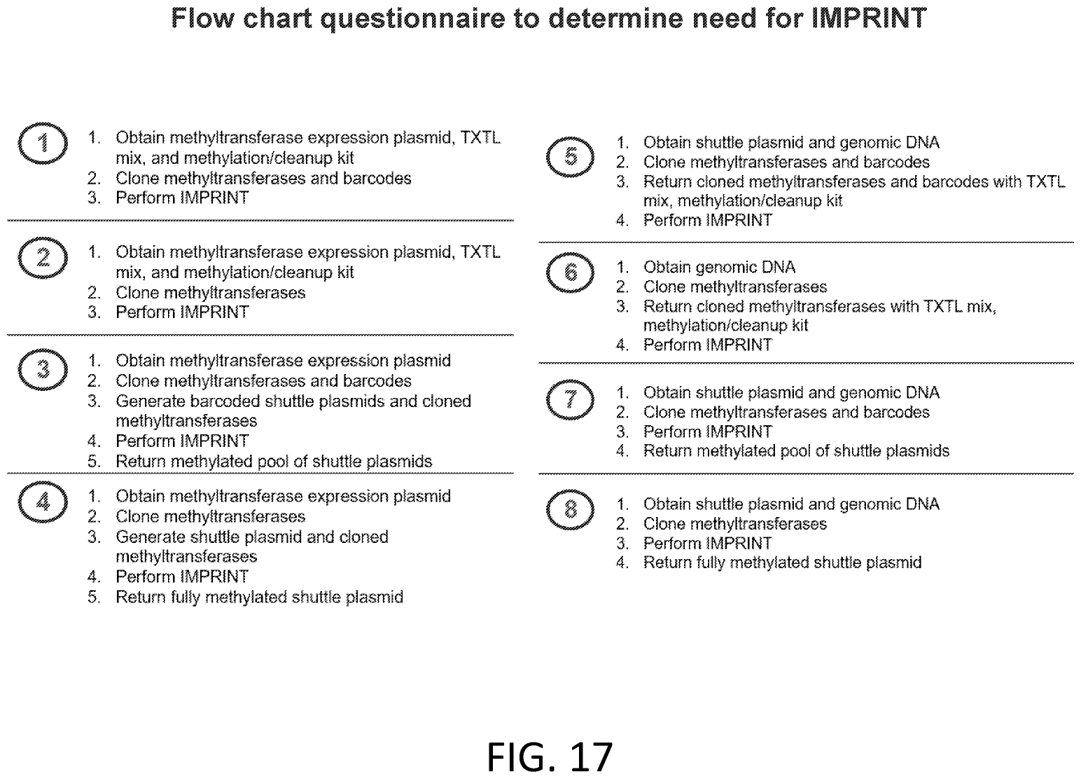

In accordance with these embodiments, a user can consult an off-site scientific professional for support in identifying a suitable methyltransferase for a desired bacterial host strain (see, e.g., FIG. 17). In some cases, an off-site scientific professional can support a user in carrying out high throughput barcoding, analyzing a lookup table, and/or conducting bioinformatics analysis. In some cases, the off-site professional can perform any of these aspects of the method without user assistance, and subsequently inform the user which methyltransferase will produce the optimal methylation pattern for a particular target DNA plasmid.

Embodiments of the present disclosure also include a method of identifying a methyltransferase compatible with a host cell. In accordance with these embodiments, the method includes: (i) generating a library of plasmid DNA constructs, wherein at least one plasmid in the library comprises a barcode associated with a candidate methyltransferase or set of methyltransferases; (ii) expressing each of the candidate methyltransferase or set of methyltransferases in a cell-free transcription-translation mixture comprising the associated barcoded plasmid construct from the library of DNA plasmid constructs, thereby methylating the associated barcoded plasmid construct; (iii) transforming the library of barcoded and methylated plasmid DNA constructs into a host cell; and (iv) determining the frequency of each of barcode and methylated plasmid DNA construct isolated from the host cell, thereby identifying the candidate methyltransferase or set of methyltransferases compatible with the host cell.

In some embodiments, the host cell is selected from the group consisting of an E. coli cell, a Bifidobacterium cell, a Bacillus cell, a Campylobacter cell, a Clostridium cell, a Corynebacterium cell, a Cyanobacterium cell, a Geobacillus cell, a Helicobacter cell, a Klebsiella cell, a Lactobacillus cell, a Mycobacterium cell, a Neisseria cell, a Paenibacillus cell, a Prevotella cell, a Pseudomonas cell, a Ralstonia cell, a Salmonella cell, a Serratia cell, a Shewanella cell, a Staphylococcus cell, a Streptococcus cell, a Vibrio cell, and a Yersinia cell, or any variants thereof. In some embodiments, the methyltransferase comprises at least one of: (i) a methyltransferase and a specificity protein associated with a Type I RM system; (ii) using a set of barcoded plasmid DNA constructs associated with a methyltransferase or a combination of methyltransferases for determining a methylation pattern and identifying a corresponding methyltransferase or combination of methyltransferases that is compatible with host cell; (iii) a methyltransferase associated with a Type III RM system; and/or (iv) an orphan methyltransferase that produces the same methylation pattern.

### 5. Materials and Methods

Standard growth conditions. E. coli and S. enterica propagation was performed in LB medium (10 g/L NaCl, 5 g/L yeast extract, 10 g/L tryptone) while being shaken at 250 rpm at 37° C., aside from E. coli KL740 which was grown at 30° C. Plasmids were maintained at the following antibiotic concentrations; ampicillin (50 μg/mL), chloramphenicol (34 μg/mL).

Bifidobacteria were routinely grown in MRS liquid broth (BD CN #288130) supplemented with 0.05% L-cysteine and MRS agar (BD CN #288210) or Reinforced Clostridial Agar (RCA, Thermo CN #CM0151B) and incubated at 37° C. in an anaerobic chamber. Tetracycline was used to maintain plasmids at a concentration of 20 μg/mL.

Plasmid generation. Plasmid pJV170 was used to readily clone new methyltransferases under P70a expression. Cloning was performed by amplifying plasmid pJV170 and host genomic DNA with primers that include homology tails, then by performing Gibson assembly according to manufacturer's protocols (NEB CN #E261IS). The assembly mix was transformed into E. coli KL740 by electroporation, and colonies were screened using colony PCR and Sanger sequencing to determine if the clone was correct.

When necessary, small insertions or mutations were inserted into plasmids using Q5 site-directed mutagenesis (NEB CN #E0554S) according to manufacturer's protocols and transforming chemically competent NEB 10-beta cells (NEB CN #C3019), where colony PCR and Sanger sequencing were again used to determine if the clone was correct.

To clone a library of barcoded E. coli-Bifidobacteria shuttle vectors, Q5 mutagenesis was performed according to manufacturer's protocols where four random nucleotides were added to the 5′ end of one of the primers. The Q5 mutagenesis mix was transformed into NEB 10-beta chemically competent cells, and individual barcodes were isolated using colony PCR and Sanger sequencing to determine the barcode sequence of each clone.

In vitro methylation of plasmid DNA. To quickly methylate DNA in vitro using IMPRINT, TXTL reactions were first set up by incubating TXTL master mix (Arbor Biosciences CN #507024) with each methyltransferase plasmid harboring the methyltransferase gene(s) to be expressed at 29° C. for 12-16 hours. The shuttle plasmid was first propagated in methyltransferase-deficient E. coli EC135 to yield an un-methylated shuttle plasmid. Then, a methylation reaction was set up (50 μL) by adding a total of 1 μL of the TXTL reaction(s) to the shuttle vector along with 1× dam methyltransferase buffer (NEB CN #M0222) and 640 μM AdoMet (NEB CN #B9003), and incubating at 37° C. for 2 hours. Following methylation, the reaction was first treated with 100 μg/mL Proteinase K and incubated at 50° C. for 30 minutes then with 100 μg/mL RNase A and incubated at 37° C. for 1 hour to remove impurities from the TXTL mix. Finally, the shuttle plasmid was purified using column purification (Zymo Research CN #D4013) according to manufacturer protocols.

Electroporation. Electroporation of S. enterica was performed as follows: an overnight culture was diluted 1:20 in fresh LB media and grown until the OD₆₀₀=0.6. Then, cells were harvested by centrifugation at 5,000 rpm and 4° C., before being washed twice with 25 mL of ice-cold 10% glycerol. Harvested cells were then suspended in 0.5 mL ice-cold 10% glycerol. 0.1 mL of cell suspension was added to 1-mm electroporation cuvettes with 50-100 ng of plasmid DNA, and the cells were electroporated at 1.8 kV, 200Ω resistance, and 25 μF. 0.9 mL of pre-warmed LB broth was then added to the electroporated cells, and a recovery was set up for 1 hour at 37° C. before cells were plated on LB agar supplemented with chloramphenicol. Colonies were counted after overnight incubation at 37° C.

To transform B. breve and B. longum by electroporation, cells were first made electrocompetent by adapting a previously published protocol⁵⁰. In short, 4 mL of an overnight culture was added to 50 mL of MRS supplemented with 1% glucose and 0.05% L-cysteine, and cells were grown at 37° C. in an anaerobic chamber until the OD₆₀₀ reached 0.6 (B. breve) or 1.0 (B. longum). Then, cells were harvested by centrifugation at 4,500 rpm and 4° C., and washed twice with 25 mL of ice-cold wash buffer (0.5 M sucrose, 1 mM ammonium citrate, pH 6.0) then once more with 1 mL of ice-cold wash buffer before resuspending the cells in 0.375 mL ice-cold wash buffer. To perform electroporation, 90 μL of the cell suspension was added to 1-mm electroporation cuvettes with 200-500 ng of plasmid DNA, and electroporation was performed at 2.0 kV, 200Ω resistance, and 25 μF. For B. longum, the cells were incubated with the plasmid DNA on ice for at least 5 minutes before performing electroporation. After electroporation, 0.9 mL of pre-warmed Reinforced Clostridial Medium (RCM) was added to the cells and a recovery was set up for 3 hours at 37° C. in an anaerobic chamber. Finally, cells were plated on Reinforced Clostridial Agar (RCA) supplemented with tetracycline, and colonies were counted after 2-3 days of incubation at 37° C.

HT-IMPRINT. To determine the optimal methylation pattern required for transformation of different Bifidobacteria strains, 4-nt barcodes were cloned into the E. coli-Bifidobacterium shuttle plasmid. Then, 2-3 barcodes were assigned to each combination of methyltransferases for a given strain. IMPRINT reactions were performed to methylate the barcoded shuttle plasmids with each combination of the methyltransferases present in the strain. The purified, barcoded shuttle vectors from the IMPRINT reactions were then pooled together and transformed into the Bifidobacteria strain, and cells were both plated on RCA supplemented with tetracycline and diluted in MRS broth supplemented with 0.05% L-cysteine and tetracycline. After 2-3 days, the transformed plasmids were prepped from the back-diluted cultures, or from the grown colonies by first adding 1 mL PBS to the agar plates to resuspend colonies. In both cases, the plasmid was used as a template to amplify a segment of the plasmid harboring the barcode. The PCR amplicon was submitted for Amplicon-EZ Sequencing performed by Genewiz (genewiz.com/en/Public/Senvices/Next-Generation-Sequencing/Amplicon-Sequencing-Services/Amplicon-EZ). The number of reads mapping to each barcode was counted within each.fastq.gz file using the command zgrep-c “CTGCNNNN” *.fastq.gz for pJV420_U barcodes or zgrep-c “GCTTNNNN” *.fastq.gz for pJV420_D barcodes. From there, the median barcode count (if using three barcodes per sample) or average barcode count (if using two barcodes per sample) was calculated for each methylation sample in the transformed cells and in the pool control. The ratio of each sample in the transformed cells compared to the pool control was compared to the ratio of total reads in the transformed cells relative to the pool control to calculate the extent of barcode enrichment or depletion.

Statistical analyses. A student's t-test was utilized to determine statistical significance in DNA transformation from different MTases. In all cases, the t-test was a two-sample, equal variance dataset with a two-tailed distribution.

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

### 6. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

While physical barriers such as the cell wall can interfere with transformation, arguably the most common barrier is posed by restriction-modification (R-M) systems. These systems are one of a growing set of defenses but represent the most prevalent across bacteria. To confer immunity. R-M systems rely on DNA methyltransferases (MTases) that methylate genomic DNA with a unique methylation pattern using S-adenosyl methionine (SAM) as a methyl donor as well as restriction endonucleases (REases) that cleave DNA with foreign patterns. Four types have been defined based on the need for a specificity protein to guide the MTase and REase (Type I), the MTase and REase acting independently (Type II), the MTase and REase forming a complex capable of both methylation and restriction (Type III), and the REase cleaving methylated DNA (Type IV). Transformed DNA possessing the wrong methylation pattern undergoes extensive cleavage, resulting in a massive drop in the number of transformed colonies. As a further challenge, bacteria often possess not one but multiple R-M systems conferring unique methylation patterns that can vary even between strains.

Recreating these patterns or mutating the short recognition sites have proven to be an effective way to restore transformation, in some cases radically boosting transformation. However, existing approaches have been heavily constrained in their ability to fully reproduce these patterns in a rapid and comprehensive means (FIG. 18). For example, approaches involving in vitro methylation of DNA with purified MTases or expressing the MTases in a plasmid-propagating strain of E. coli are laborious and suffer from methylation-induced cytotoxicity, typically limiting their use to one or two DNA MTases that often provide incomplete protection. Separately, an approach to identify and mutate MTase sites requires methylome sequencing followed by resynthesizing entire DNA constructs, where many of these mutations could interfere with plasmid maintenance or the expression and function of the encoded constructs. Therefore, new approaches are needed to simplify how R-M systems are circumvented across bacteria.

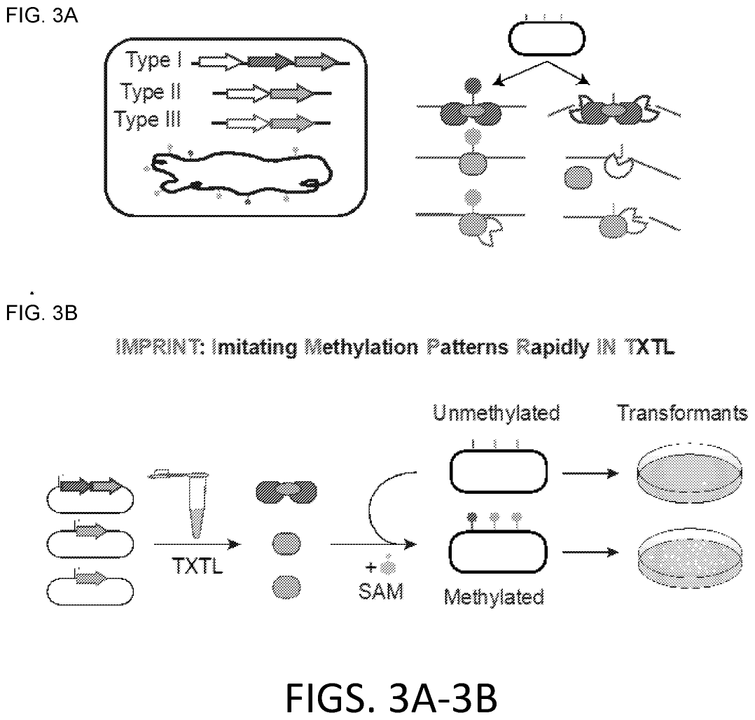

To tackle this challenge, experiments were conducted to evaluate TXTL as a distinct means to overcome DNA restriction. TXTL recapitulates transcription and translation in a lysate or solution of purified components, allowing the functional expression of RNA and protein in minutes to hours without the need for cell culturing or protein purification. A TXTL-based pipeline was developed in which DNA MTases identified in a host bacterium (e.g., through bioinformatics or listed on the REBASE database) are expressed and combined with DNA in a methylation reaction, and the resulting DNA is purified and transformed into the host. DNA lacking any methylation (e.g., PCR product, plasmid DNA extracted from a MTase-free strain) serves as the starting point to immediately circumvent Type IV R-M systems that cleave methylated DNA. The resulting pipeline was termed IMPRINT (Imitating Methylation Patterns Rapidly IN TXTL) (FIG. 3A). With IMPRINT, methylation by multiple MTases, including Type I MTases that also require expressing a specificity protein, is straightforward and completed in under a day. Any cytotoxicity concerns are also minimized due to the absence of replicating cells for plasmid methylation.

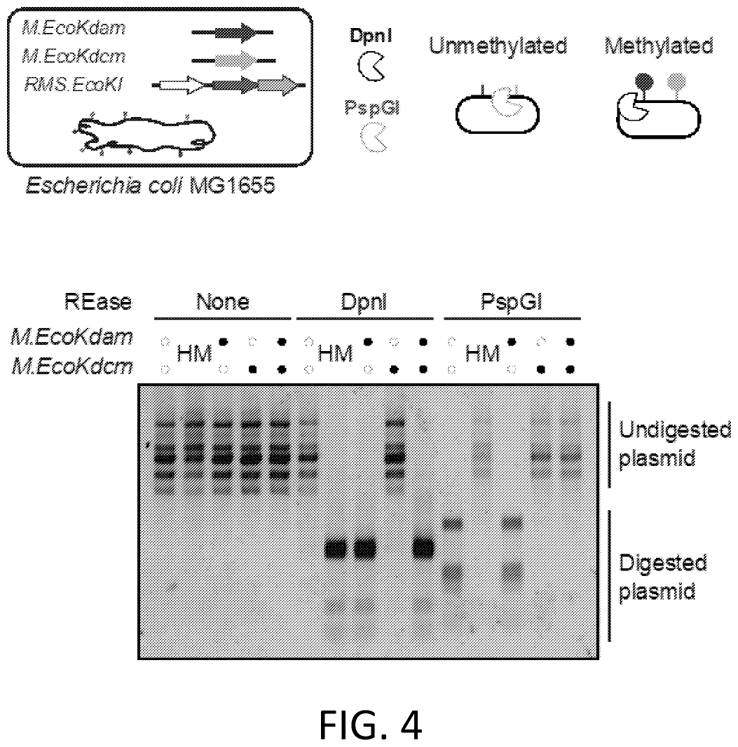

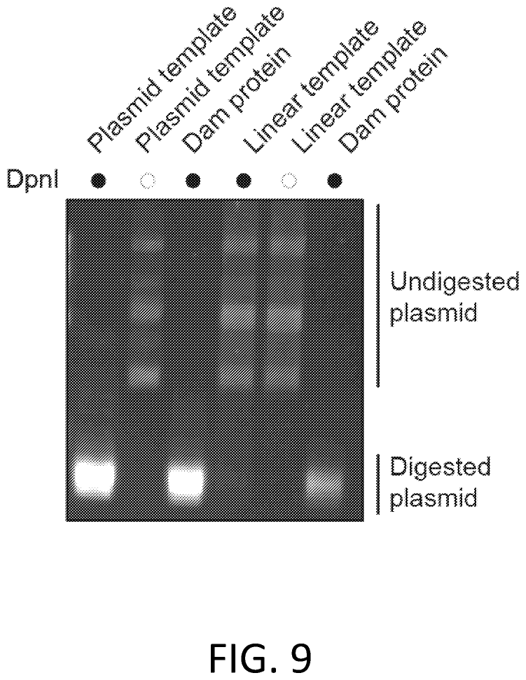

IMPRINT was initially prototyped by expressing MTases from E. coli. Standard laboratory strains of E. coli (e.g. MG1655) possess two orphan DNA MTases, Dam (M.EcoKdam) and Dcm (M.EcoKdcm), which respectively methylate adenine in GATC and the second cytosine in CC(A/T)GG. To assess methylation, the restriction enzyme DpnI was used, which cuts GATC when methylated by Dam, and PspGI, which cuts CC(A/T)GG unless it is methylated by Dcm (FIG. 3B). Each MTase was encoded in an overexpression plasmid and expressed in TXTL. The expressed MTases were then incubated individually or together with the E. coli plasmid pJV24 containing 10 Dam recognition sites and 7 Dcm recognition sites. After completing IMPRINT, DpnI digested the plasmid incubated with Dam or both Dam and Dem, while PspGI was unable to digest the plasmid incubated with Dcm or both Dam and Dcm (FIG. 4). Similar digestion patterns were obtained with the plasmid extracted from E. coli MG1655, which expresses both MTases. Complete digestion with DpnI and complete protection from PspGI affirmed efficient methylation with both MTases. Less-efficient methylation was achieved with Dam encoded on a linear DNA construct (FIG. 9), establishing that linear constructs can also be used. These results show that IMPRINT can be used to methylate plasmid DNA with MTases expressed in TXTL.

### Example 2

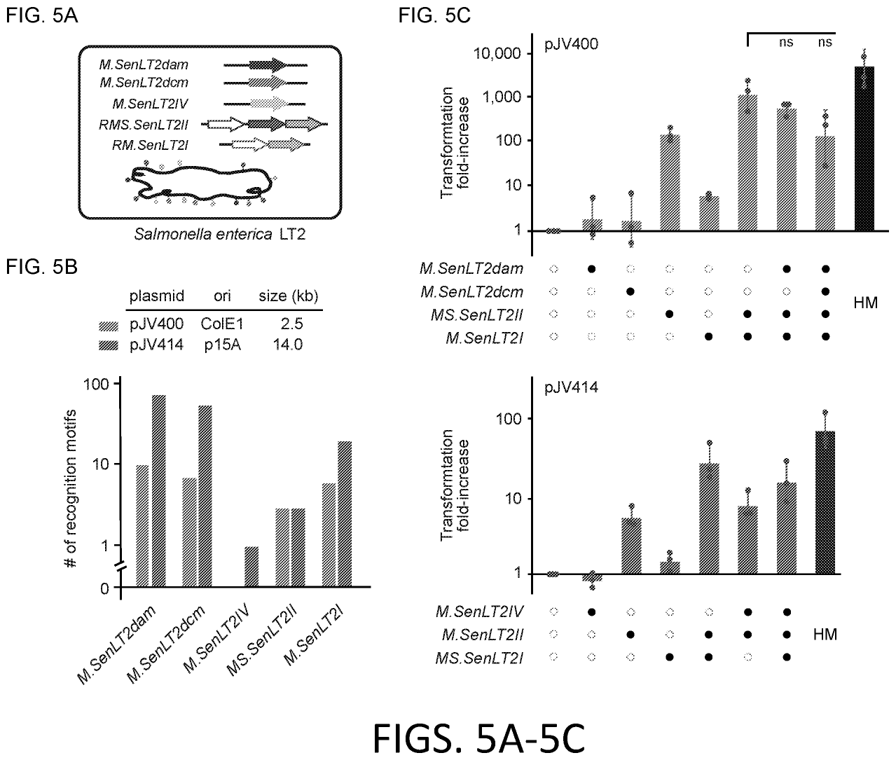

The next major question was whether DNA methylated through IMPRINT could boost transformation. The LT2 strain of the gram-negative pathogen Salmonella enterica was used, which is related to E. coli but restricts E. coli DNA through its set of well-characterized R-M systems. As listed on REBASE, the associated MTases include two orphan MTases Dan (M.SenLT2dam) and Dem (M.SenLT2dcm) homologous to those in E. coli, another orphan MTase (M.SenLT2IV), a Type I MTase requiring a specificity protein (MS.SenLT2II), and a Type III MTase (M.SenLT2I) (FIG. 5A). The five MTases were cloned into expression plasmids, with the MTase and specificity genes for the Type I R-M system cloned as an operon. Two transformation plasmids were further selected (the 2.5 kb JV400 and the 14-kb JV414) harboring a different number of recognition sites for each MTase and different modes of replication (FIG. 5B). For the three MTases with compatible REases (M.SenLT2dam with DpnI, M.SenLT2dcm with PspGI, and M.SenLT2I V with NsiI), methylation by each MTase was confirmed based on affected cleavage by the corresponding REase (FIG. 10A-10D). Methylation by the Type III MTase was confirmed by cloning a REase site at the methylation site (FIG. 10D). Either pJV400 or pJV414 methylated with different MTase combinations was then electroporated into LT2 (FIG. 5C). Compared to the unmethylated plasmid, none of the orphan MTases significantly enhanced transformation. In contrast, the Type I MTase and specificity protein provided a large boost in transformation (131-fold for JV400, 6-fold for JV414), while the Type III MTase provided a smaller but significant boost (6-fold for JV400, 1.5-fold for JV414). Importantly, combining both MTases in the same methylation reaction resulted in a much larger boost (1,000-fold for JV400, 32-fold for JV414) that approached the transformation boost for plasmid DNA extracted from LT2 possessing the strain's complete methylation pattern (4,300-fold for JV400, 86-fold for JV414). Therefore, IMPRINT can utilize multiple MTases from various R-M systems to boost DNA transformation.

### Example 3

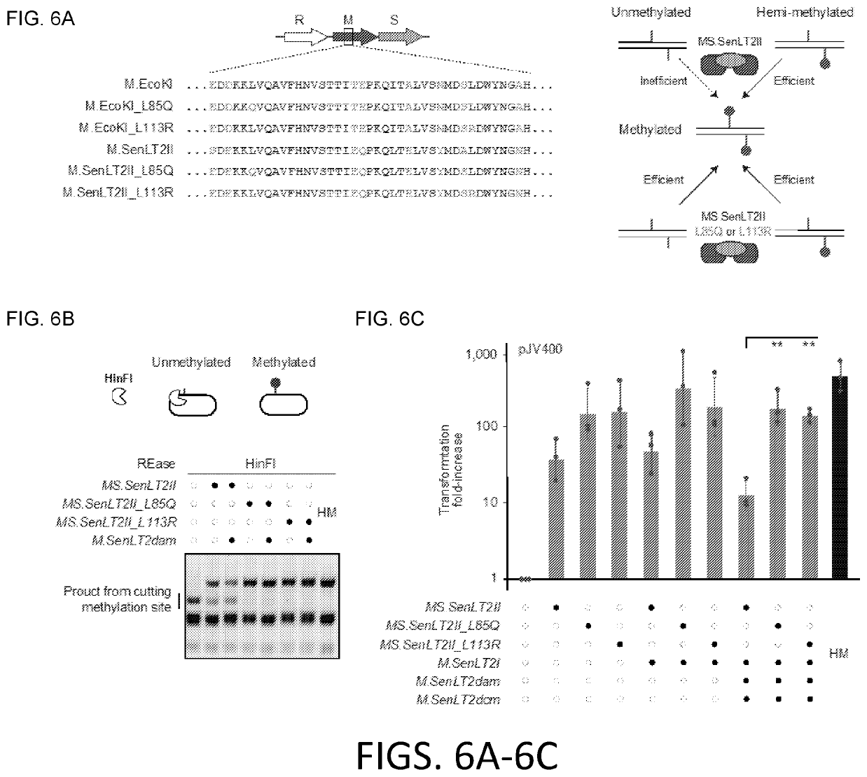

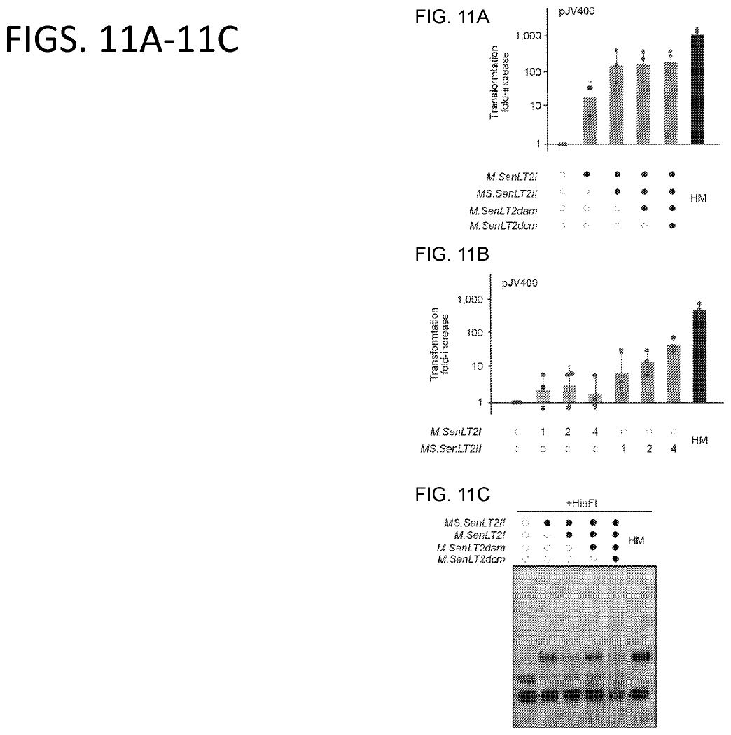

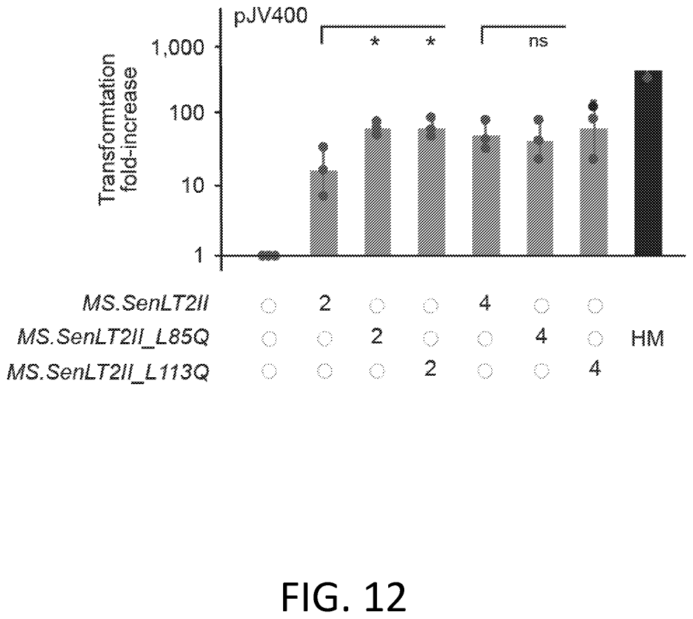

It was observed that including any of the orphan MTases with the Types I and III MTases reduced transformation (FIG. 5C). As this reduction could pose a limitation for bacteria harboring large numbers of R-M systems, experiments were conducted to evaluate whether transformation was reduced and if it could be restored. It was first found that methylating plasmid DNA in a series of methylation reactions rather than a one-pot reaction restored transformation, suggesting that one of the MTases exhibited reduced activity (FIG. 11A) in the presence of the other MTases. When evaluating the rate of methylation with the Type I and III MTases, it was noticed that the Type I MTase required a longer reaction time to achieve complete methylation based on transformation into LT2 (FIG. 11B). Similarly, the Type 1 MTase provided incomplete protection against cleavage of an introduced overlapping REase site, particularly in the presence of other MTases (FIG. 11C). One explanation for incomplete protection is that this subtype of MTases (Type IA) prefers hemimethylated substrates, slowing methylation and allowing the other MTases to compete for available SAM. Fortuitously, prior work isolated mutants of the Type I MTase in E. coli (M.EcoKI) that readily accepted unmethylated substrates (FIG. 6A). Introducing two of these mutations into the Type I MTase from LT2 (FIG. 6B), it was found that either mutation improved protection against HinFI cleavage from partial to complete even when including a second MTase (FIG. 6C). The mutations also enhanced transformation into LT that was maintained when simultaneously methylating with Dam, Dem, and the Type III MTase from LT2 (FIG. 6D) and allowed for shorter reaction times (FIG. 12). These results show that the Type I MTases can be a bottleneck in DNA methylation, although mutating the MTase to remove the preference for hemimethylated substrates can enhance transformation and allow the inclusion of other MTases in the methylation reaction.

### Example 4

By applying different MTase combinations using IMPRINT, it was determined that the Type I and III MTases contributed to enhanced DNA transformation in S. enterica LT2, while the orphan MTases played no role in enhanced transformation. For bacteria with a large number of R-M systems or much more laborious and intensive transformation procedures, testing the MTases individually or in combinations would be a long and involved process. Therefore, experiments were conducted to create a high-throughput version of IMPRINT that tests different combinations of MTases in a single transformation (FIG. 7). To reach this goal, a setup was devised in which a set of short barcodes are introduced into the shuttle plasmid. Several barcodes are then associated with one combination of MTases in case a given barcode introduces or removes a methylation site. The full set of barcodes representing all tested MTase combinations are then transformed into the host bacterium, and the transformed cells are plated or back-diluted into liquid medium. After isolating plasmid DNA from pooled colonies or liquid culture, the relative frequency of each barcode can be quantified by next-generation amplicon sequencing. The extent of barcode enrichment compared to the untransformed library would represent the boost in transformation provided by the associated MTase combination. This approach was termed high-throughput IMPRINT (HT-IMPRINT).

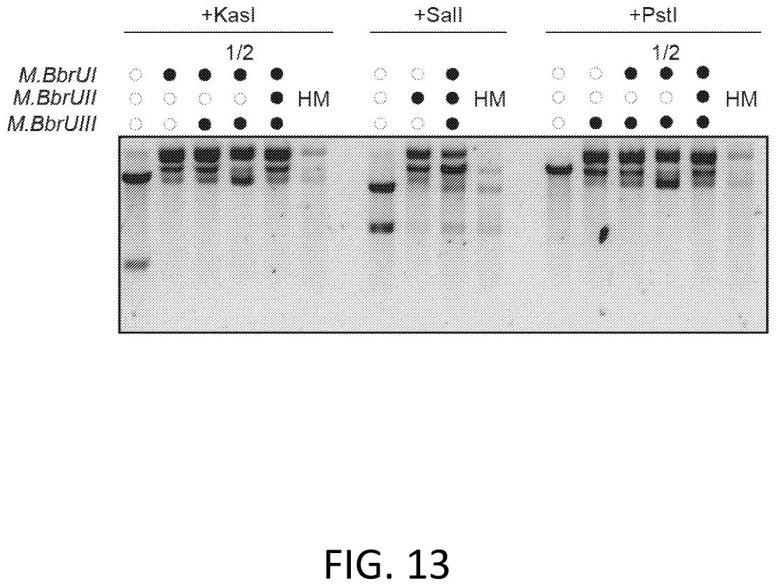

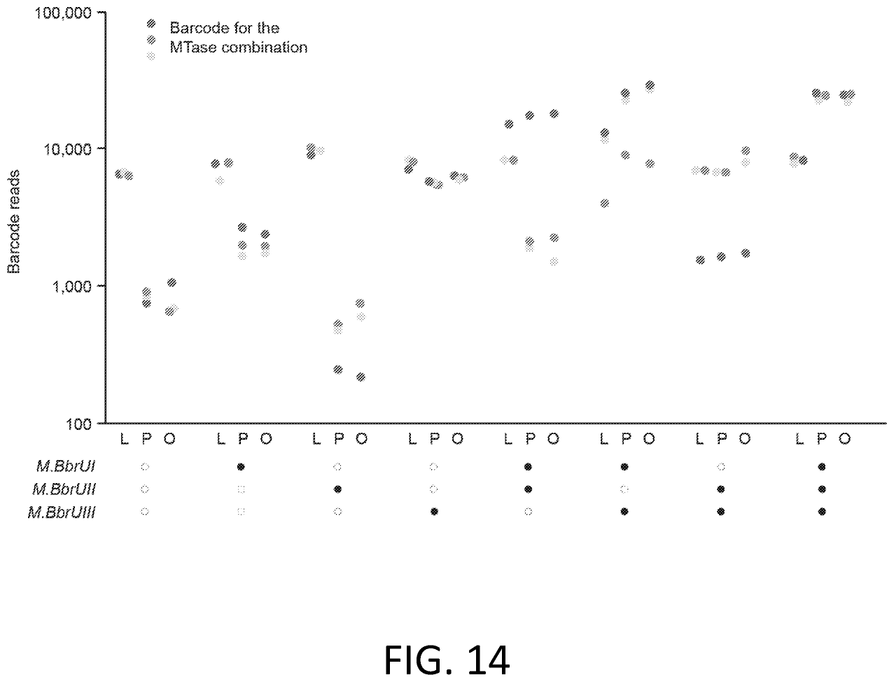

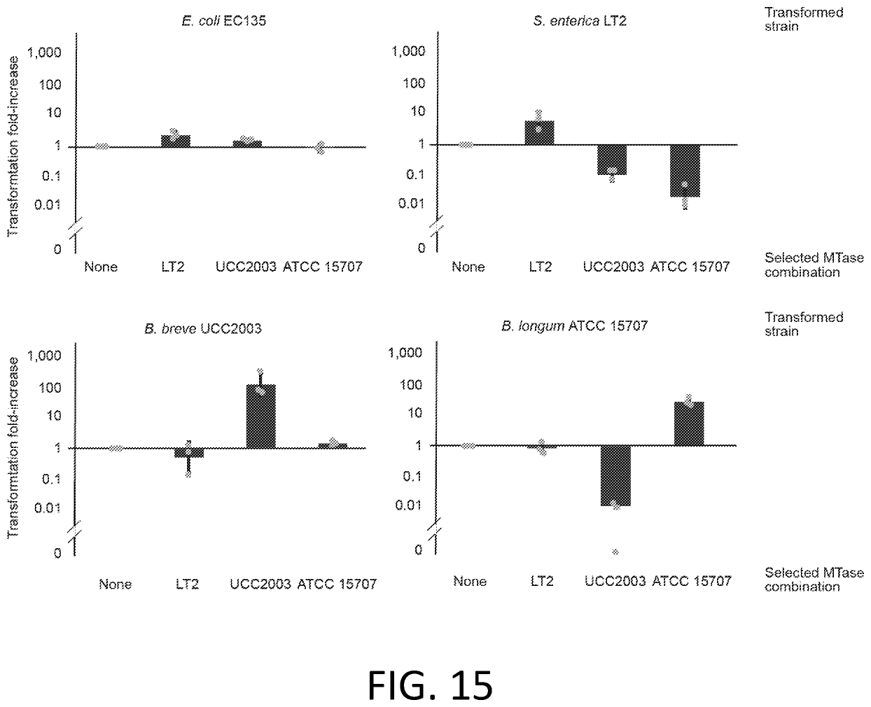

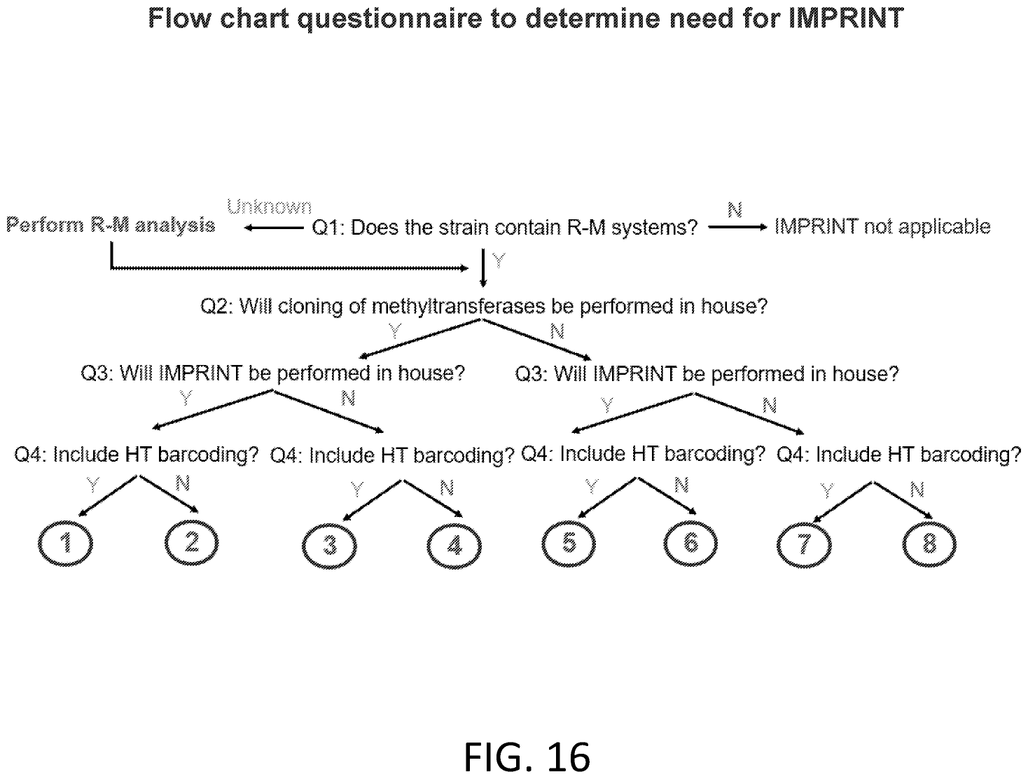

To apply HT-IMPRINT and move beyond bacteria related to E. coli, Bifidobacteria was used. These gram-positive bacteria are common constituents of the human digestive tract, and many species are used as probiotics. These bacteria are also strict anaerobes with more involved transformation procedures, possess a wide variety of R-M systems, and have proven difficult to transform. Initial experiments were conducted using one strain, Bifidobacterium breve UCC2003, which harbors three characterized Type II R-M systems listed on REBASE and previously shown to interfere with DNA transformation (FIG. 8A). The three associated MTases (M.BbrUI, M.BbrUII, M.BbrUIII) were cloned into expression plasmids for IMPRINT, which were confirmed to methylate DNA in TXTL based on blocked cleavage of associated REases (FIG. 13). A total of 24 unique barcodes were then introduced into the Bifidobacterium-E. coli shuttle plasmid JV420 to cover the eight possible combinations of MTases. Following IMPRINT with each MTase combination, the pooled plasmids were transformed into B. breve UC2003, and transformed DNA was recovered from pooled colonies or liquid culture followed by amplicon sequencing. The output from both approaches revealed that M.BbrUI and M.BbrUIII individually boosted transformation, with M.BbrUIII having a much stronger effect. Furthermore, combining all three MTases provided the greatest boost in transformation (FIG. 8B). Notably, there was very little variation among the three barcodes from each sample as a result of the screen (FIG. 14). Testing individual MTase combinations with IMPRINT yielded the same trend, where similarly a high boost in transformation over unmethylated DNA was achieved when combining M.BbrUI and M.BbrUIII (128-fold) and combining all three MTases (140-fold) (FIG. 8C). Importantly, the extent of transformation matched that for the shuttle plasmid extracted from B. breve UCC2003 (FIG. 8D), showing that DNA restriction was fully circumvented using IMPRINT. The results also matched the previously published role of M.BbrUIII in DNA restriction, although our results show that including other MTases leads to an additive boost in transformation.

### Example 5

Experiments were also conducted to apply HT-IMPRINT to Bifidobacterium longum ATCC 15707. This strain contains two R-M systems listed on REBASE, where the Type II system encodes two different MTases (M₁.Blo1217ORF1038, M₂.Blo1217ORF1038) and the Type I system encodes an MTase with two different specificity proteins (MS₁S₂.Blo1217ORF1481) (FIG. 8D). This strain has also proven extremely difficult to transform, and the R-M systems remain uncharacterized. Because of the lack of characterization, each MTase or MTase/specificity protein pair was cloned and then immediately subjected to HT-IMPRINT using the pJV420 shuttle plasmid, selecting 12 MTase combinations covered with 24 barcodes. The resulting transformed cells did not grow in liquid culture once antibiotics were added, while 25 colonies were obtained following plating-both outcomes likely due to sub-optimal selection conditions (FIG. 8E). The colonies were associated with a mix of MTase combinations, where the most common MTases were M₁.Blo1217ORF1038, M₂.Blo12170RF1038, and/or MS₂.Blo1217ORF1481. Testing M₁M₂.Blo1217ORF1038 and MS₂.Blo1217ORF1481 individually and together using IMPRINT supported the output from HT-IMPRINT, where the greatest boost in transformation compared to unmethylated DNA came from the paired combination (74-fold) followed by M₁M₂.Blo1217ORF1038 (49-fold) and then MS₂.Blo12170RF1481 (6-fold) (FIG. 8F). The boost in transformation could not be compared to host-methylated DNA, as negligible DNA could be extracted from this strain. Overall, HT-IMPRINT allowed us to determine an effective combination of MTases for a recalcitrant strain of Bifidobacteria.

HT-IMPRINT revealed the most consequential MTase combinations for different Bifidobacteria. In some cases, the methylation pattern for one strain would boost transformation in other strains, easing the process of transforming diverse bacteria. Experiments were then conducted to evaluate how the best MTase combination identified via IMPRINT in one strain impacts transformation in other strains. The best MTase combinations for S. enterica LT2, B. breve UCC2003, and B. longum ATCC 15707 were selected and these were applied to the Bifidobacterium-E. coli shuttle plasmid JV420 that can be propagated in all strains (FIG. 8G). Transforming this plasmid with each methylation pattern into each strain, it was found that transformation was highest when the MTase combination was matched with the originating host strain (FIG. 8H). Interestingly, unmethylated DNA transformed more efficiently than DNA methylated with a pattern not from the host strain. The largest drop occurred using the B. breve methylation pattern to transform B. longum, showing that the methylation pattern of related species can be detrimental. Taking these data together, the best methylation pattern mapped to the originating strain, while transformation could be hindered by using an incorrect methylation pattern. The species-specific methylation pattern needed to enhance DNA transformation highlights the need for approaches tailored to individual bacteria, as afforded by IMPRINT.

Taken together, the results of the present disclosure demonstrate that IMPRINT offers an effective means to recreate the pattern of DNA methylation in a host bacterium using TXTL, thereby boosting transformation of the methylated DNA into the host. Furthermore, applying HT-IMPRINT allowed the identification of the best MTase combination in a single transformation, simplifying subsequent transformation efforts. By avoiding cell culturing and protein purification required by traditional approaches, IMPRINT greatly accelerates the time from obtaining constructs for each MTase to achieving improved transformation. The results of the present disclosure further showed that methylation could be enhanced with mutations to Type I MTases that relieve the preference for hemimethylated substrates.

The final product of IMPRINT is purified DNA that can be transformed through different means, including electroporation, chemical transformation, and natural transformation, as well as packaged into nanoparticles. While IMPRINT utilizes naked DNA that is not readily compatible with cell-based delivery approaches such as conjugation or phage delivery, HT-IMPRINT identified a minimal set of important MTases to express in the donor/packaging strain. Outside of R-M systems, novel antiphage defense systems continue to be discovered throughout bacteria that could also impact DNA transformation. Systems that rely on DNA methylation (e.g., BREX, DISARM) or other forms of DNA chemical modification can be incorporated into IMPRINT to allow transformed DNA to circumvent an even wider assortment of bacterial defenses. Regardless of the specific delivery mode or type of MTase, increased transformation by IMPRINT provides for efficient genetic manipulation and harnesses the rich diversity of bacteria for sustainable chemical production, enhancing food production, and applying cell-based biosensors and therapeutics. IMPRINT is also useful for studying the role of R-M systems in host defense and gene regulation, creating opportunities to explore the functional roles played by DNA MTases in diverse bacteria.

Sequences. The various embodiments of the present disclosure described herein may include one or more of the sequences referenced below, which can be found in the corresponding sequence listing.

M.EcoKI (FIG. 6A):

EDDKKLVQAVFHNVSTfITEPKQITALVSNMDSLDSLDWYNGAH (SEQ ID NO: 1)

M.EcoKI_L85Q (FIG. 6A):

EDDKKQVQAVFHNVSTTITEPKQITALVSNMDSLDSLDWYNGAH (SEQ ID NO: 2)

M.EcoKI_LI 13R (FIG. 6A):

EDDKKLVQAVFHNVSTTITEPKQITALVSNMDSLDSRDWYNGAH (SEQ ID NO: 3)

M.SenLT2II (FIG. 6A):

SDEKKLVQAVFHNVSTTIEQPKQITELVSYMDALDSRDWYNGNH (SEQ ID NO: 4)

M.SenLT2II_L85Q (FIG. 6A):

EDEKKQVQAVFHNVSTTIEQPKQITELVSYMDSLDSRDWYNGNH (SEQ ID NO: 5)

M.SenLT2I1_L113R (FIG. 6A):

EDEKKLVQAVFHNVSTTIEQPKQITELVSYMDSRDSRDWYNGNH (SEQ ID NO: 6)

Primer sequences used in the various embodiments of the present disclosure are provided in Table 1 below.

TABLE 1
Primer sequences.

| Primer | Stock | Sequence |
|---|---|---|
| pJV420_U_F | JV605 | NNNNAGGCATGCAAGCTTGGCGTA (SEQ ID NO: 7) |
| pJV420_U_R | JV606 | GCAGGTCGACTCTAGAGGAT (SEQ ID NO: 8) |
| pJV420_D_F | JV706 | GGCGTAATCATGGTCATAGC (SEQ ID NO: 9) |
| pJV420_D_R | JV707 | NNNNAAGCTTGCATGCCTGCAG (SEQ ID NO: 10) |
| HT_sequence_F | JV647 | GGCTTGTGCCACAACGGTGT (SEQ ID NO: 11) |
| HT_sequence_R | JV643 | GGCAGTGAGCGCAACGCAAT (SEQ ID NO: 12) |

Barcode sequences used in the various embodiments of the present disclosure are provided in Table 2 below.

TABLE 2
Barcode sequences used for HT-IMPRINT in B. breve UCC2003.

| Barcode Numbers | Barcode Sequences | Methylation Combination |
|---|---|---|
| D1-D2-D3 | GTGG-GAAG-GCTG (SEQ ID NO: 13) | Empty |
| D4-D5-D6 | GGAG-ACGA-TCGA (SEQ ID NO: 14) | M.BbrUI |
| D7-D8-D9 | CACA-GCAC-TAAA (SEQ ID NO: 15) | M.BbrUII |
| D10-D11-D12 | CCCG-CCAT-ACAT (SEQ ID NO: 16) | M.BbrUIII |
| D13-D14-D15 | CGTC-CAAG-GGAA (SEQ ID NO: 17) | M.BbrUI + M.BbrUII |
| D16-D17-D18 | AAAG-TGCC-GGAT (SEQ ID NO: 18) | M.BbrUI + M.BbrUIII |
| D19-D20-D21 | AAAC-TGAA-CTCG (SEQ ID NO: 19) | M.BbrUII + M.BbrUIII |
| D22-D23-D24 | TGTC-CTGA-CGCC (SEQ ID NO: 20) | M.BbrUI + M.BbrUII + M.BbrUIII |

Barcode sequences used in the various embodiments of the present disclosure are provided in Table 3 below.

TABLE 3
Barcode sequences used for HT-IMPRINT in B. longum ATCC15707.

| Barcode | | |
|---|---|---|
| Numbers | Barcode Sequences | Methylation Combination |
| U1-D1 | ATCT-GTGG (SEQ ID NO: 21) | Empty |
| U2-D2 | CGTT-GAAG (SEQ ID NO: 22) | MS2.Blo1217ORF1481 |
| U3-D3 | TCGT-GCTG (SEQ ID NO: 23) | M1.Blo1217ORF1038 |
| U4-D4 | ACTT-GGAG (SEQ ID NO: 24) | M2.Blo1217ORF1038 |
| U5-D5 | AATA-ACGA (SEQ ID NO: 25) | MS.S2.Blo1217ORF1481 |
| U6-D6 | GTGT-TCGA (SEQ ID NO: 26) | MS2.Blo1217ORF1481 + |
| | | M1.Blo1217ORF1038 |
| U7-D7 | TCTA-CACA (SEQ ID NO: 27) | MS2.Blo1217ORF1481 + |
| | | M2.Blo1217ORF1038 |
| U8-D8 | CCTC-GCAC (SEQ ID NO: 28) | M1M2.Blo1217ORF1038 |
| U9-D9 | GGTT-TAAA (SEQ ID NO: 29) | MS1S2.Blo1217ORF1481 + |
| | | S1.Blo1217ORF1481 |
| U10-D10 | CGCA-CCCG (SEQ ID NO: 30) | MS1S2.Blo1217ORF1481 + |
| | | M2.Blo1217ORF1038 |
| U11-D11 | CTTA-CCAT (SEQ ID NO: 31) | MS2.Blo1217ORF1481 + |
| | | M1M2.Blo1217ORF1038 |
| U12-D12 | TTAG-ACAT (SEQ ID NO: 32) | MS1S2.Blo1217ORF1481 + |
| | | M1M2.Blo1217ORF1038 |

Plasmid constructs used in the various embodiments of the present disclosure are provided in Table 4 below.

TABLE 4
Plasmid Constructs.

| Plasmid | Description | Plasmid map |
|---|---|---|
| pJV170 | P70a-GFP plasmid to clone methy | benchling.com/s/seq- |
| | ltransferases | QKS9eCV32deK7RTaY4iX?m=slm- |
| | | whD895YlrMKPbOOScN11 |
| pJV441 | P70a-T7 RNA polymerase expression | benchling.com/s/seq- |
| | construct | BVJEFTHnKix0NxHtCqF5?m=slm- |
| | | JfM0yu4RGmDL521e3oVq |
| pJV24 | M.EcoKdam and M.EcoKdcm validation | benchling.com/s/seq- |
| | | LBM9s4EYOtkBiERMJrnD?m=slm- |
| | | bG8trK8GcJHMgWm1ZLN6 |
| pJV400 | LT2 shuttle vector ColE1 origin | benchling.com/s/seq- |
| | | jfs3oAJOBfy2Bc8HP4cR?m=slm- |
| | | jtyKyagVktbSNtsY3e0c |
| pJV414 | LT2 shuttle vector p15A origin | benchling.com/s/seq- |
| | | p4ST6tBYAVdcSKkQ46wE?m=slm- |
| | | a5ysOUDUujoscjrEyAug |
| pJV420 | Bifidobacteria-E. coli shuttle vector | benchling.com/s/seq- |
| | | mvth5uDmtTwQVcRWen6X?m=slm- |
| | | Hrq3dwA61YnoAeuDXZji |
| pJV302 | M.EcoKdam expression construct | benchling.com/s/seq- |
| | | zIRYJPvWpX0m1VL88ybl?m=slm- |
| | | MsaVRc9GH8xDsrbU44sh |
| pJV593 | M.EcoKdcm expression construct | benchling.com/s/seq- |
| | | CZlWHrWAIqPCZOiO5fOO?m=slm- |
| | | HWkasz34xucCDAVjcn1J |
| pJV388 | M.SenLT2dam expression construct | benchling.com/s/seq- |
| | | YHeAIUMK2jmo3kdQw1Gs?m=slm- |
| | | uLrEOWFutqrJfp8FVQfP |
| pJV389 | M.SenLT2dem expression construct | benchling.com/s/seq- |
| | | MhdR9tlyxUzsLfyQZLTR?m=slm- |
| | | OWUZft3LhTMAt1wLOXR5 |
| pJV303 | M.SenLT2I expression construct | benchling.com/s/seq- |
| | | 7zdYwfROPjYXRXIcGDZa?m=slm- |
| | | ggslC6gkpkxxhjNNymHb |
| pJV265 | MS.SenLT2II expression construct | benchling.com/s/seq- |
| | | s5trithkW8Z9hQ36YKiR?m=slm- |
| | | Es2oD8onPFUfNBAw0faT |
| pJV505 | MS.SenLT2II_L85Q expression construct | benchling.com/s/seq- |
| | | SJQsWVAfyHmTNpSrXfz7?m=slm- |
| | | J628G3xsYNqVOBcODYd6 |
| pJV506 | MS.SenLT211_L113R expression construct | benchling.com/s/seq- |
| | | lQRAmjcvHlTuGsnPcVPU?m=slm- |
| | | uoGuOdLibfLdnUk5VjxE |
| pJV354 | M.SenLT2IV expression construct | benchling.com/s/seq- |
| | | 3AUPzdlUI6ZdfvE7k3ZA?m=slm- |
| | | N1PYpz5Hu8CSAM9sZUiO |
| pJV184 | M.SenLT2I validation plasmid | benchling.com/s/seq- |
| | | 3hL0DbE5d6y0aj6j01Sn?m=slm- |
| | | pEslkMAGvUIpVLloKoDg |
| pJV412 | MS.SenLT2II validation plasmid | benchling.com/s/seq- |
| | | t3ioJvLnqn78yGxFgIks?m=slm- |
| | | XMAzWaAo6CRRyG1XCPS2 |
| pJV447 | M.SenLT2IV validation plasmid | benchling.com/s/seq- |
| | | egWA09ByPTMeBa60xTPq?m=slm- |
| | | G0kVaR8frDgtXnLAlspd |
| pJV457 | M.BbrUI expression construct | benchling.com/s/seq- |
| | | oTIPpDXWTbC3t6eymOah?m=slm- |
| | | rv8Di1WfiCZGwe8R1tzc |
| pJV476 | M.BbrUlI expression construct | benchling.com/s/seq- |
| | | keb6VaRGtou3cU0Fthtr?m=slm- |
| | | y9XVvvDfPIYhtgRWUv6O |
| pJV461 | M.BbrUIII expression construct | benchling.com/s/seq- |
| | | XhzqE9uH4FNkngCv4RC3?m=slm- |
| | | Blpa3SGxKPe4mY39jNle |
| pJV544 | M1.Blo1217ORF1038 expression constrict | benchling.com/s/seq- |
| | | 10CE0ukWudaJ9pLOefem?m=slm- |
| | | Kwc8mWF9jOAWUUxeGAYM |
| pJV466 | M2.Blo1217ORF1038 expression construct | benchling.com/s/seq- |
| | | ZJsAzSjsy657HZ5kSEtU?m=slm- |
| | | sXLa8vVT7rhNdw6VVtIK |
| pJV465 | MS2.Blo1217ORF1481 expression construct | benchling.com/s/seq- |
| | | WmNO9xhyJBerBZupl4WF?m=slm- |
| | | ttx2FmWCs3BfncqA23UJ |
| pJV543 | S1.Blo1217ORF1481 expression construct | benchling.com/s/seq- |
| | | OCPQx3imuI6k5SKnx635?m=slm- |
| | | o7leh9KDFVLwUXJvR5Xg |
| pJV420_U | Bifidobacteria-E. coli plasmid with | benchling.com/s/seq- |
| | upstream barcode | MzUfYaewZ3e0LpOdZaDH?m=slm- |
| | | TnCP6CBCC9J0YMaPhe8u |
| pJV420_D | Bifidobacteria-E. coli plasmid with | benchling.com/s/seq- |
| | downstream barcode | Bh6YanVKjG1XTz8W3G5a?m=slm- |
| | | s2bAbKefEtCGjwnYj04E |

What is claimed is:

1. A composition for methylating target DNA for transformation into a host cell, the composition comprising:
a cell-free transcription-translation mixture; and
at least one expression construct encoding one or more components of a methylation complex;
wherein the methylation complex replicates at least a portion of a methylation pattern used by the host cell on the target DNA.

2. The composition of claim 1, wherein replicating the methylation pattern used by the host cell results in enhanced transformation efficiency.

3. The composition of claim 1, wherein replicating the methylation pattern used by the host cell bypasses the host cell's restriction modification (RM) system.

4. The composition of any of claims 1 to 3, wherein the host cell is a bacterial cell.

5. The composition of claim 4, wherein the bacterial cell is selected from the group consisting of an E. coli cell, a Bifidobacterium cell, a Bacillus cell, a Campylobacter cell, a Clostridium cell, a Corynebacterium cell, a Cyanobacterium cell, a Fusobacterium cell, a Geobacillus cell, a Helicobacter cell, a Klebsiella cell, a Lactobacillus cell, a Mycobacterium cell, a Neisseria cell, a Paenibacillus cell, a Prevotella cell, a Pseudomonas cell, a Ralstonia cell, a Salmonella cell, a Serratia cell, a Shewanella cell, a Staphylococcus cell, a Streptococcus cell, a Vibrio cell, and a Yersinia cell, or any variants thereof.

6. The composition of any of claims 1 to 5, wherein the cell free transcription-translation mixture is derived from bacterial cell lysate.

7. The composition of any of claims 1 to 6, wherein at least one component of the cell free transcription-translation mixture is purified.

8. The composition of any of claims 1 to 7, wherein the one or more components of the methylation complex comprises at least one of:
(i) a methyltransferase, and a specificity protein associated with a Type I RM system in the host cell;
(ii) a methyltransferase associated with a Type II RM system in the host cell;
(iii) a methyltransferase associated with a Type III RM system in the host cell; and/or
(iv) an orphan methyltransferase.

9. The composition of any of claims 1 to 8, wherein the one or more components of the methylation complex comprises at least one methyltransferase.

10. The composition of claim 9, wherein the at least one methyltransferase is derived from the host cell.

11. The composition of claim 9, wherein the at least one methyltransferase is mutated.

12. The composition of claim 11, wherein the mutated methyltransferase comprises a mutation that enhances methylation of the target DNA.

13. The composition of claim 11, wherein the mutation occurs in the N-terminal domain of a Type IA methyltransferase.

14. The composition of claim 11, wherein the mutated methyltransferase comprises a mutation that enhances methylation of unmethylated DNA.

15. The composition of any of claims 1 to 14, further comprising S-Adenosyl methionine (SAM) as a methyl donor.

16. The composition of any of claims 1 to 15, further comprising a suitable methylation buffer.

17. The composition of any of claims 1 to 16, further comprising RNase A and/or Proteinase K.

18. A kit for methylating target DNA for transformation into a host cell, the kit comprising:
a cell-free transcription-translation mixture; and
at least one expression construct encoding one or more components of a methylation complex;
wherein the methylation complex replicates at least a portion of a methylation pattern used by the host cell on the target DNA.

19. The kit of claim 18, wherein the one or more components of the methylation complex comprises at least one of:
(i) a methyltransferase and a specificity protein associated with a Type I RM system;
(ii) a methyltransferase associated with a Type II RM system;
(iii) a methyltransferase associated with a Type III RM system; and/or
(iv) an orphan methyltransferase.

20. The kit of claim 18, wherein the one or more components of the methylation complex comprises a mutated methyltransferase, wherein the mutation enhances recognition of unmethylated DNA.

21. The kit of any of claims 18 to 20, further comprising one or more of: (i)S-Adenosyl methionine (SAM) as a methyl donor; (ii) a suitable methylation buffer; (iii) RNase A; and/or (iv) Proteinase K.

22. The kit of any of claim 18 to 21, further comprising a set of barcoded plasmid DNA constructs for determining a methylation pattern in a host cell.

23. The kit of any of claims 18 to 22, further comprising a lookup table comprising a list of methyltransferases associated with a host cell.

24. The kit of claim 23, wherein the host cell is selected from the group consisting of an E. coli cell, a Bifidobacteria cell, a Bacillus cell, a Campylobacter cell, a Clostridium cell, a Fusobacterium cell, a Helicobacter cell, a Mycobacterium cell, a Neisseria cell, a Pseudomonas cell, a Salmonella cell, a Staphylococcus cell, a Streptococcus cell, a Vibrio cell, and a Yersinia cell, or any variants thereof.

25. A method of methylating a target DNA for transforming into a host cell comprising expressing one or more components of a methylation complex in a cell-free transcription-translation mixture comprising the target DNA, wherein the methylation complex replicates at least a portion of a methylation pattern used by the host cell on the target DNA.

26. The method of claim 25, wherein replicating the methylation pattern used by the host cell bypasses the host cell's restriction modification (RM) system and enhances transformation efficiency.

27. The method of claim 25 or claim 26, wherein the host cell is selected from the group consisting of an E. coli cell, a Bifidobacterium cell, a Bacillus cell, a Campylobacter cell, a Clostridium cell, a Corynebacterium cell, a Cyanobacterium cell, a Fusobacterium cell, a Geobacillus cell, a Helicobacter cell, a Klebsiella cell, a Lactobacillus cell, a Mycobacterium cell, a Neisseria cell, a Paenibacillus cell, a Prevotella cell, a Pseudomonas cell, a Ralstonia cell, a Salmonella cell, a Serratia cell, a Shewanella cell, a Staphylococcus cell, a Streptococcus cell, a Vibrio cell, and a Yersinia cell, or any variants thereof.

28. The method of any one of claims 25 to 27, wherein the one or more components of the methylation complex comprises at least one of:
(i) a methyltransferase, and a specificity protein associated with a Type I RM system in the host cell;
(ii) a methyltransferase associated with a Type II RM system in the host cell;
(iii) a methyltransferase associated with a Type III RM system in the host cell; and/or
(iv) an orphan methyltransferase.

29. The method of any one of claims 25 to 28, wherein the one or more components of the methylation complex comprises a mutated methyltransferase, wherein the mutation enhances recognition of unmethylated DNA.

30. The method of any one of claims 25 to 29, further comprising isolating and/or purifying the methylated target DNA prior to transforming into the host cell.

31. The method of any one of claims 25 to 30, further comprising determining the one or more components of a methylation complex that are compatible with the host strain.

32. The method of claim 31, wherein determining the one or more components of a methylation complex that are compatible with the host strain comprises at least one of:
(i) consulting a lookup table comprising a list of methyltransferases associated with the host cell;
(ii) using a set of barcoded plasmid DNA constructs for determining a methylation pattern used in the host cell; and/or
(iii) conducting bioinformatics analysis.

33. A method of identifying a methyltransferase compatible with a host cell, the method comprising:
(i) generating a library of plasmid DNA constructs, wherein at least one plasmid in the library comprises a barcode associated with a candidate methyltransferase or set of methyltransferases;
(ii) expressing each of the candidate methyltransferase or set of methyltransferases in a cell-free transcription-translation mixture comprising the associated barcoded plasmid construct from the library of DNA plasmid constructs, thereby methylating the associated barcoded plasmid construct;
(iii) transforming the library of barcoded and methylated plasmid DNA constructs into a host cell; and
(iv) determining the frequency of each of barcode and methylated plasmid DNA construct isolated from the host cell, thereby identifying the candidate methyltransferase or set of methyltransferases compatible with the host cell.

34. The method of claim 33, wherein the host cell is selected from the group consisting of an E. coli cell, a Bifidobacterium cell, a Bacillus cell, a Campylobacter cell, a Clostridium cell, a Corynebacterium cell, a Cyanobacterium cell, a Geobacillus cell, a Helicobacter cell, a Klebsiella cell, a Lactobacillus cell, a Mycobacterium cell, a Neisseria cell, a Paenibacillus cell, a Prevotella cell, a Pseudomonas cell, a Ralstonia cell, a Salmonella cell, a Serratia cell, a Shewanella cell, a Staphylococcus cell, a Streptococcus cell, a Vibrio cell, and a Yersinia cell, or any variants thereof.

35. The method of claim 33 or claim 34, wherein the candidate methyltransferase or set of methyltransferases comprises at least one of:
(i) a methyltransferase and a specificity protein associated with a Type I RM system;
(ii) a methyltransferase associated with a Type II RM system;
(iii) a methyltransferase associated with a Type III RM system; and/or
(iv) an orphan methyltransferase.


SEQUENCE LISTING (32 sequences)

SEQ ID NO: 1 - protein, 44 aa, Escherichia coli
EDDKKLVQAVFHNVSTTITEPKQITALVSNMDSLDSLDWYNGAH

SEQ ID NO: 2 - protein, 44 aa, Escherichia coli
EDDKKQVQAVFHNVSTTITEPKQITALVSNMDSLDSLDWYNGAH

SEQ ID NO: 3 - protein, 44 aa, Salmonella enterica
EDDKKLVQAVFHNVSTTITEPKQITALVSNMDSLDSRDWYNGAH

SEQ ID NO: 4 - protein, 44 aa, Salmonella enterica
SDEKKLVQAVFHNVSTTIEQPKQITELVSYMDALDSRDWYNGNH

SEQ ID NO: 5 - protein, 44 aa, Salmonella enterica
EDEKKQVQAVFHNVSTTIEQPKQITELVSYMDSLDSRDWYNGNH

SEQ ID NO: 6 - protein, 44 aa, Salmonella enterica
EDEKKLVQAVFHNVSTTIEQPKQITELVSYMDSRDSRDWYNGNH

SEQ ID NO: 7 - DNA, 24 bp, synthetic construct
  source 1..24: mol_type = other DNA
nnnnaggcatgcaagcttggcgta

SEQ ID NO: 8 - DNA, 20 bp, synthetic construct
  source 1..20: mol_type = other DNA
gcaggtcgactctagaggat

SEQ ID NO: 9 - DNA, 20 bp, synthetic construct
  source 1..20: mol_type = other DNA
ggcgtaatcatggtcatagc

SEQ ID NO: 10 - DNA, 22 bp, synthetic construct
  source 1..22: mol_type = other DNA
nnnnaagcttgcatgcctgcag

SEQ ID NO: 11 - DNA, 20 bp, synthetic construct
  source 1..20: mol_type = other DNA
ggcttgtgccacaacggtgt

SEQ ID NO: 12 - DNA, 20 bp, synthetic construct
  source 1..20: mol_type = other DNA
ggcagtgagcgcaacgcaat

SEQ ID NO: 13 - DNA, 12 bp, synthetic construct
  source 1..12: mol_type = other DNA
gtgggaaggctg

SEQ ID NO: 14 - DNA, 12 bp, synthetic construct
  source 1..12: mol_type = other DNA
ggagacgatcga

SEQ ID NO: 15 - DNA, 12 bp, synthetic construct
  source 1..12: mol_type = other DNA
cacagcactaaa

SEQ ID NO: 16 - DNA, 12 bp, synthetic construct
  source 1..12: mol_type = other DNA
cccgccatacat

SEQ ID NO: 17 - DNA, 12 bp, synthetic construct
  source 1..12: mol_type = other DNA
cgtccaagggaa

SEQ ID NO: 18 - DNA, 12 bp, synthetic construct
  source 1..12: mol_type = other DNA
aaagtgccggat

SEQ ID NO: 19 - DNA, 12 bp, synthetic construct
  source 1..12: mol_type = other DNA
aaactgaactcg

SEQ ID NO: 20 - DNA, 12 bp, synthetic construct
  source 1..12: mol_type = other DNA
tgtcctgacgcc

SEQ ID NO: 21-32 - skipped (12 entries)